(19) United States
(12) Patent Application Publication
(10) Pub. No.: US 2012/0276049 A1
(43) Pub. Date: Nov. 1, 2012

(54) Methods and compositions for amelioration of autoimmune disease using fusion proteins of anti-dendritic cell receptor antibody to peptide sequences

(75) Inventors: Joel N. H. Stern, Seaford, NY (US); Jack L. Strominger, Cambridge, MA (US)

(73) Assignee: President and Fellows of Harvard College, Cambridge, MA (US)

(21) Appl. No.: 13/442,057

(22) Filed: Apr. 9, 2012

Related U.S. Application Data
(63) Continuation of application No. PCT/US10/51962, filed on Oct. 8, 2010.
(60) Provisional application No. 61/249,715, filed on Oct. 8, 2009.

(51) Int. Cl. A61K 39/395; G01N 33/577; A61P 37/06; A61P 7/06; A61P 7/00; A61P 27/02; A61P 3/10; A61P 25/00; A61P 21/04; A61P 17/06; A61P 29/00; A61P 19/02; A61P 31/04; A61P 31/12; A61K 38/21; A61P 35/00; A61P 37/02; C07K 19/00

(52) U.S. Cl. 424/85.6; 530/387.3; 424/134.1; 435/7.24; 424/85.4

(57) ABSTRACT

Compositions that are fusion proteins of antibodies to dendritic cell receptors, exemplified by anti-DEC205 and anti-33D1 antibodies, to peptide sequences that are immunosuppressive or tolerogenic are provided for treatment of autoimmune diseases such as multiple sclerosis. Also provided are pharmaceutical compositions including the fusion proteins, as well as therapeutic methods for administering the fusion proteins.

### RELATED APPLICATION

This application is a continuation of PCT/US2010/051962 filed Oct. 8, 2010, which claims benefit of provisional, application Ser. No. 61/249,715 filed Oct. 8, 2009, each of which are hereby incorporated herein by reference in their entireties.

### GOVERNMENT SUPPORT

The invention was made in part with support from grant number R01AI49524 from the National Institutes of Health. The government has certain rights in the invention.

### TECHNICAL FIELD

The invention relates to methods of use and compositions of fusion of designed peptides to an antibody protein for treatment of demyelinating autoimmune disease such as multiple sclerosis (MS).

### BACKGROUND

Multiple sclerosis (MS) is a T cell-mediated chronic autoimmune inflammatory disease characterized by prominent lymphocyte and macrophage infiltration into the white matter, inflammatory demyelination of neuronal axons, and axonal loss in the human central nervous system (Hafler, D. A. et al. 1989 Immunol Today 10:104; Zamvil, S. S. et al. 1990 Annu Rev Immunol 8:579). This pathology is associated with neurological dysfunctions such as paralysis, sensory deficit and visual problems. The cause of the disease is unknown, but both environmental and genetic factors are important. HLA-DR2 (DRA*0101, DRB1*1501), an allele of a multi-gene family encoding antigen receptors known as MHC class II proteins, is present at increased frequency in northern European patients with MS (Spielman, R. S. et al. 1982 Epidemiol Rev 4:45; Hillert, J. et al. 1994 J Neuroimmunol 50:95; Oksenberg, J. R. et al. 1993 Jama 270:2362).

Myelin basic protein (MBP) is thought to be a major target antigen in the pathogenesis of MS. Particularly, T cell reactivity to the immunodominant MBP 85-99 epitope is found in subjects carrying HLA-DR2, a genetic marker for susceptibility to MS. HLA-DR2-restricted MBP-specific T cells are clonally expanded and activated in MS patients (Wucherpfennig, K. W. et al. 1991 Immunol Today 12:277; Markovic-Plese, S. et al. 1995 J Immunol 155:982; Kerlero de Rosbo, N. et al. 1997 Eur J Immunol 27:3059; Tsuchida, T. et al. 1994 Proc Natl Acad Sci USA 91:10859; Illes, Z. et al. 1999 J Immunol 162:1811; Allegretta, M. et al. 1990 Science 247:718). Furthermore, a complex of HLA-DR2/MBP is detected in the CNS plaques of these patients (Krogsgaard, M. et al. 2000 J Exp Med 191:1395). Residues for binding to HLA-DR2 and for TCR recognition of the MBP 85-99 epitope have been determined (Wucherpfennig, K. W. et al. 1994 J Exp Med 179:279; Smith, K. J. et al. 1998 J Exp Med 188:1511).

MS has been linked to the autoimmune response of T cells to myelin self-antigens presented by HLA-DR2 with which MS is genetically associated, and MBP is a major candidate autoantigen in this disease. A random amino acid copolymer Copolymer 1 or Cop1, Copaxone®, Glatiramer Acetate, poly(Y, E, A, K)n, [YEAK] as well as two additional synthetic copolymers [poly (F,Y,A,K)n or FYAK, and poly (V,W,A,K)n or VWAK] also form complexes with HLA-DR2 (DRA/DRB1*1501) and compete with MBP85-99 for binding (Teitelbaum, D. et al. 1971 Eur J Immunol 1:242; Fridkis-Hareli, M. et al. 1998 J Immunol 160:4386; Fridkis-Hareli, M. et al. 2002 J Clin Invest 109:1635).

Therapeutic approaches to MS utilizing cytokines, copolymers, dimers of class II MHC-peptide complexes, peptide antigens that induce anergy, vaccination with TCR and an altered peptide ligand have been explored (APL; Fridkis-Hareli, M. et al. 2001 Hum Immunol 62:753; Gaur, A. et al. 1992 Science 258:1491; Leonard, J. P. et al. 1996 Ann N Y Acad Sci 795:216; Nicholson, L. B. et al. 1995 Immunity 3:397; Fridkis-Hareli, M. et al. 2002 J Clin Invest 109:1635; Ruiz, P. J. 2001. J Immunol 167:2688; Goodkin, D. E. et al. 2000 Neurology 54:1414). Copolymer 1, the only approved drug known to reduce MBP-specific T cell responses, reduces the relapse rate by 30% in relapsing-remitting forms of MS (Teitelbaum, D. et al. 1971 Eur J Immunol 1:242; Teitelbaum, D. et al. 1973 Eur J Immunol 3:273; Teitelbaum, D. et al. 1974 Clin Immunol Immunopathol 3:256; Aharoni, R. et al. 1993 Eur J Immunol 23:17; Bornstein, M. B. et al. 1987 N Engl J Med 317:408; Johnson, K. P. et al. 1995 Neurology 45:1268; Johnson, K. P. et al. 1998 Neurology 50:701).

Additional more effective agents and methods are needed to treat MS and other autoimmune diseases.

### SUMMARY

An embodiment of the invention provides a composition including a fusion protein having an amino acid sequence of a monoclonal antibody specific for binding a dendritic cell receptor protein and an amino acid sequence of an immunosuppressive peptide or a tolerogenic peptide. Dendritic cell receptor proteins are exemplified by mannose receptors and toll-like receptors. For example, the dendritic cell receptor protein is selected from at least one of the group: DEC205, CLEC9A and 33D1.

In some embodiments, the amino acid sequence of the immunosuppressive peptide includes EKPKVEAYKAAAAPA (SEQ ID NO: 1). For example, the amino acid sequence of the peptide is selected from the group of: EKPK (SEQ ID NO: 2), KPKV (SEQ ID NO: 3), PKVE (SEQ ID NO: 4), KVEA (SEQ ID NO: 5), VEAY (SEQ ID NO: 6), EAYK (SEQ ID NO: 7), AYKA (SEQ ID NO: 8), YKAA (SEQ ID NO: 9), KAAA (SEQ ID NO: 10), AAAA (SEQ ID NO: 11), AAAP (SEQ ID NO: 12), AAPA (SEQ ID NO: 13), EKPKV (SEQ ID NO: 14), KPKVE (SEQ ID NO: 15), PKVEA (SEQ ID NO: 16), KVEAY (SEQ ID NO: 17), VEAYK (SEQ ID NO: 18), EAYKA (SEQ ID NO: 19), AYKAA (SEQ ID NO: 20), YKAAA (SEQ ID NO: 21), KAAAA (SEQ ID NO: 22), AAAAP (SEQ ID NO: 23), AAAPA (SEQ ID NO: 24), EKPKVE (SEQ ID NO: 25), KPKVEA (SEQ ID NO: 26), PKVEAY (SEQ ID NO: 27), KVEAYK (SEQ ID NO: 28), VEAYKA (SEQ ID NO: 29), EAYKAA (SEQ ID NO: 30), AYKAAA (SEQ ID NO: 31), YKAAAA (SEQ ID NO: 32), KAAAAP (SEQ ID NO: 33), AAAAPA (SEQ ID NO: 34), EKPKVEA (SEQ ID NO: 35), KPKVEAY (SEQ ID NO: 36), PKVEAYK (SEQ ID NO: 37), KVEAYKA (SEQ ID NO: 38), VEAYKAA (SEQ ID NO: 39), EAYKAAA (SEQ ID NO: 40), AYKAAAA (SEQ ID NO: 41), YKAAAAP (SEQ ID NO: 42), KAAAAPA (SEQ ID NO: 43), EKPKVEAY (SEQ ID NO: 44), KPKVEAYK (SEQ ID NO: 45), PKVEAYKA (SEQ ID NO: 46), KVEAYKAA (SEQ ID NO: 47), VEAYKAAA (SEQ ID NO: 48), EAYKAAAA (SEQ ID NO: 49), AYKAAAAP (SEQ ID NO: 50), YKAAAAPA (SEQ ID NO: 51), EKPKVEAYK (SEQ ID NO: 52), KPKVEAYKA (SEQ ID NO: 53), PKVEAYKAA (SEQ ID NO: 54), KVEAYKAAA (SEQ ID NO: 55), VEAYKAAAA (SEQ ID NO: 56), EAYKAAAAP (SEQ ID NO: 57), AYKAAAAPA (SEQ ID NO: 58), EKPKVEAYKA (SEQ ID NO: 59), KPKVEAYKAA (SEQ ID NO: 60), PKVEAYKAAA (SEQ ID NO: 61), KVEAYKAAAA (SEQ ID NO: 62), VEAYKAAAAP (SEQ ID NO: 63), EAYKAAAAPA (SEQ ID NO: 64), EKPKVEAYKAA (SEQ ID NO: 65), KPKVEAYKAAA (SEQ ID NO: 66), PKVEAYKAAAA (SEQ ID NO: 67), KVEAYKAAAAP (SEQ ID NO: 68), VEAYKAAAAPA (SEQ ID NO: 69), EKPKVEAYKAAA (SEQ ID NO: 70), KPKVEAYKAAAA (SEQ ID NO: 71), PKVEAYKAAAAP (SEQ ID NO: 72), KVEAYKAAAAPA (SEQ ID NO: 73), EKPKVEAYKAAAA (SEQ ID NO: 74), KPKVEAYKAAAAP (SEQ ID NO: 75), PKVEAYKAAAAPA (SEQ ID NO: 76), EKPKVEAYKAAAAP (SEQ ID NO: 77), and KPKVEAYKAAAAPA (SEQ ID NO: 78).

In an embodiment, the tolerogenic peptide of the composition is an encephalitogenic peptide derived from at least one protein from the group: proteolipid protein (PLP), myelin basic protein (MBP), and myelin oligodendrocyte protein (MOG). For example, the tolerogenic peptide has the amino acid sequence HSLGKWLGHPNKF (SEQ ID NO: 80).

The peptide in various embodiments has a length of at least four amino acid residues. For example, the peptide has a length of at least 15 amino acid residues. The composition can include a pharmaceutically acceptable salt, carrier or buffer. The composition further can include an additional therapeutic agent. For example, the therapeutic agent is selected from the group of: a cytotoxic agent, an immunosuppressive agent, and a chemotherapeutic agent.

The fusion protein is specific for binding the dendritic cell receptor protein which is, for example, a DEC205 receptor or a 33D1 receptor. For example, the fusion protein has an immunomodulatory function. In certain embodiments, the immunomodulatory function is a tolerogenic or an immunosuppressive function. For example, the immunomodulatory function involves inhibition of MHC class II interaction with T cells.

The composition in certain embodiments includes a unit dose effective for treatment of a subject for an autoimmune condition. In general, the autoimmune condition is a demyelinating condition. The demyelinating condition in certain embodiments is multiple sclerosis (MS). In various embodiments, the autoimmune condition is a cell mediated disease, for example is mediated by a T cell or a natural killer (NK) cell, or is an antibody mediated disease. Exemplary autoimmune conditions include: autoimmune hemolytic anemia, autoimmune oophoritis, autoimmune thyroiditis, autoimmune uveoretinitis, Crohn's disease, chronic immune thrombocytopenic purpura, colitis, contact sensitivity disease, diabetes mellitus, Graves disease, Guillain-Barre's syndrome, Hashimoto's disease, idiopathic myxedema, myasthenia gravis, psoriasis, pemphigus vulgaris, rheumatoid arthritis, and systemic lupus erythematosus.

An embodiment of the present invention provides a kit for treating a subject having an autoimmune disease, the kit including a fusion of amino acid sequence. Further, the kit in various embodiments includes a pharmaceutically acceptable buffer, a container and instructions for use.

An embodiment of the present invention provides a method for treating a subject for an autoimmune disease involving steps of: providing a fusion protein having a first amino acid sequence from a monoclonal antibody that specifically binds a dendritic cell receptor protein and a second amino acid sequence from an immunosuppressive peptide or a tolerogenic peptide; contacting the subject with a composition involving the fusion protein; and observing a reduction or an elimination of at least one symptom of the autoimmune disease. For example, the antibody binds the dendritic cell receptor protein selected from at least one of the group: DEC205, CLEC9A and 33D1. For example, the second amino acid sequence is EKPKVEAYKAAAAPA (SEQ ID NO: 1). In further examples, the second amino acid sequence is selected from the group of: EKPK (SEQ ID NO: 2), KPKV (SEQ ID NO: 3), PKVE (SEQ ID NO: 4), KVEA (SEQ ID NO: 5), VEAY (SEQ ID NO: 6), EAYK (SEQ ID NO: 7), AYKA (SEQ ID NO: 8), YKAA (SEQ ID NO: 9), KAAA (SEQ ID NO: 10), AAAA (SEQ ID NO: 11), AAAP (SEQ ID NO: 12), AAPA (SEQ ID NO: 13), EKPKV (SEQ ID NO: 14), KPKVE (SEQ ID NO: 15), PKVEA (SEQ ID NO: 16), KVEAY (SEQ ID NO: 17), VEAYK (SEQ ID NO: 18), EAYKA (SEQ ID NO: 19), AYKAA (SEQ ID NO: 20), YKAAA (SEQ ID NO: 21), KAAAA (SEQ ID NO: 22), AAAAP (SEQ ID NO: 23), AAAPA (SEQ ID NO: 24), EKPKVE (SEQ ID NO: 25), KPKVEA (SEQ ID NO: 26), PKVEAY (SEQ ID NO: 27), KVEAYK (SEQ ID NO: 28), VEAYKA (SEQ ID NO: 29), EAYKAA (SEQ ID NO: 30), AYKAAA (SEQ ID NO: 31), YKAAAA (SEQ ID NO: 32), KAAAAP (SEQ ID NO: 33), AAAAPA (SEQ ID NO: 34), EKPKVEA (SEQ ID NO: 35), KPKVEAY (SEQ ID NO: 36), PKVEAYK (SEQ ID NO: 37), KVEAYKA (SEQ ID NO: 38), VEAYKAA (SEQ ID NO: 39), EAYKAAA (SEQ ID NO: 40), AYKAAAA (SEQ ID NO: 41), YKAAAAP (SEQ ID NO: 42), KAAAAPA (SEQ ID NO: 43), EKPKVEAY (SEQ ID NO: 44), KPKVEAYK (SEQ ID NO: 45), PKVEAYKA (SEQ ID NO: 46), KVEAYKAA (SEQ ID NO: 47), VEAYKAAA (SEQ ID NO: 48), EAYKAAAA (SEQ ID NO: 49), AYKAAAAP (SEQ ID NO: 50), YKAAAAPA (SEQ ID NO: 51), EKPKVEAYK (SEQ ID NO: 52), KPKVEAYKA (SEQ ID NO: 53), PKVEAYKAA (SEQ ID NO: 54), KVEAYKAAA (SEQ ID NO: 55), VEAYKAAAA (SEQ ID NO: 56), EAYKAAAAP (SEQ ID NO: 57), AYKAAAAPA (SEQ ID NO: 58), EKPKVEAYKA (SEQ ID NO: 59), KPKVEAYKAA (SEQ ID NO: 60), PKVEAYKAAA (SEQ ID NO: 61), KVEAYKAAAA (SEQ ID NO: 62), VEAYKAAAAP (SEQ ID NO: 63), EAYKAAAAPA (SEQ ID NO: 64), EKPKVEAYKAA (SEQ ID NO: 65), KPKVEAYKAAA (SEQ ID NO: 66), PKVEAYKAAAA (SEQ ID NO: 67), KVEAYKAAAAP (SEQ ID NO: 68), VEAYKAAAAPA (SEQ ID NO: 69), EKPKVEAYKAAA (SEQ ID NO: 70), KPKVEAYKAAAA (SEQ ID NO: 71), PKVEAYKAAAAP (SEQ ID NO: 72), KVEAYKAAAAPA (SEQ ID NO: 73), EKPKVEAYKAAAA (SEQ ID NO: 74), KPKVEAYKAAAAP (SEQ ID NO: 75), PKVEAYKAAAAPA (SEQ ID NO: 76), EKPKVEAYKAAAAP (SEQ ID NO: 77), and KPKVEAYKAAAAPA (SEQ ID NO: 78).

The tolerogenic peptide in certain embodiments of the method is an encephalitogenic peptide. For example, the tolerogenic encephalitogenic peptide is derived from at least one protein selected from the group of: proteolipid protein (PLP), myelin basic protein (MBP) and myelin oligodendrocyte protein (MOG). For example, the tolerogenic encephalitogenic peptide has the amino acid sequence HSLGKWLGHPNKF (SEQ ID NO: 80).

The method in certain embodiments further involves treating the autoimmune disease by targeting the fusion protein to the dendritic cells, ameliorating at least one symptom of the disease by promoting T-cell anergy and generating suppressor T cells thereby inducing tolerance.

In a related embodiment, the reduction or elimination of the symptom is observing a decrease in severity or frequency of recurrences of at least one symptom.

In certain embodiments, the method involves providing the fusion protein, for example, chemically linking the monoclonal antibody and the peptide. Alternatively, providing the fusion protein involves engineering a recombinant nucleic acid sequence having a nucleic acid sequence encoding the first amino acid sequence from a chain of the monoclonal antibody or a fragment thereof and the second amino acid sequence from the peptide; and expressing the recombinant nucleic acid sequence in cells. For example, the recombinant nucleic acid sequence encodes the first amino acid sequence of the peptide as the fusion to the first amino acid sequence of a heavy chain of the antibody C-terminus. The monoclonal antibody is produced from a hybridoma cell line.

The autoimmune disease in certain embodiments includes a demyelinating condition, for example, multiple sclerosis (MS), encephalomyelitis, or symptoms involving hardened patches in brain, spinal cord, or other areas of the nervous system. In alternative embodiments, the autoimmune disease is selected from: autoimmune hemolytic anemia, autoimmune oophoritis, autoimmune thyroiditis, autoimmune uveoretinitis, Crohn's disease, chronic immune thrombocytopenic purpura, colitis, contact sensitivity disease, diabetes mellitus, Graves disease, Guillain-Barre's syndrome, Hashimoto's disease, idiopathic myxedema, multiple sclerosis, myasthenia gravis, psoriasis, pemphigus vulgaris, rheumatoid arthritis, and systemic lupus erythematosus.

In general, the subject is a mammal. For example, the subject is a human, a rodent, a canine, an equine, a bovine or a large value agricultural animal. The rodent, for example, is a mouse with experimental allergic encephalomyelitis, or a humanized mouse. For example, the subject is the human who is a patient with MS.

An embodiment of the method provides the composition that is administering by a route selected from the group of: intravenous (i.v.), subcutaneous (s.c), intramuscular (i.m.), and intraperitoneal (i.p.).

After contacting the subject with the composition, the method in related embodiments further involves analyzing a physiological parameter of the demyelinating condition. For example, analyzing the physiological parameter is measuring reactivity of T cells from the subject to a peptide of myelin basic protein. For example, the peptide is MBP 85-99.

The method in various embodiments further includes administering an additional therapeutic agent. For example, the additional therapeutic agent is selected from the group of an antibody, an enzyme inhibitor, an antibacterial agent, an antiviral agent, a steroid, a nonsteroidal anti-inflammatory agent, an antimetabolite, a cytokine, a cytokine blocking agent, an adhesion molecule blocking agent, a soluble cytokine receptor, a sphingosine-1-phosphate receptor modulator, and a random linear amino acid copolymer composition. For example, the antibody is a humanized monoclonal antibody specific to α4-integrin. For example, the enzyme inhibitor is a type II topoisomerase inhibitor. For example, the sphingosine-1-phosphate receptor modulator is fingolimod, or Gilenya, an immunosuppressive drug derived from myriocin, a metabolite of Isaria sinclairii (Paugh, S. W. et al. 2003 FEBS Lett 554:189) In further examples, the cytokine is an interferon such as interferon-β. For example, the copolymer is selected from the group of YEAK (Copaxone®), FYAK, VWAK and VFAK.

In another embodiment the method involving the amount of the fusion protein required to induce tolerance in subjects is for example less than about 1 mg, less than about 500 μg, less than about 300 μg, or less than about 100 μg. For example, the amount is at least about 10 ng, 100 ng, 1 μg, 50 μg, 100 μg, 150 μg, 200 μg, 250 μg or 300 μg.

An embodiment of the invention herein provides a method for detecting the presence of a DEC205 receptor in a biological sample, involving contacting a biological sample with the fusion protein amino acid sequence; and detecting the fusion protein bound to the DEC205 receptor thereby detecting DEC205 receptor.

### BRIEF DESCRIPTION OF THE DRAWINGS

FIG. 1 is a drawing showing the structure of an anti-DEC205 monoclonal antibody (SEQ ID NO: 79) fused to a synthetic designed peptide, J5 (SEQ ID NO: 1) or PLP139-151 (SEQ ID NO: 80). The peptide herein is fused to a C-terminus of the heavy chain of anti-DEC205 antibody.

FIG. 2 is a set of line graphs showing data obtained from treatment of subjects for experimental autoimmune encephalomyelitis (EAE). Data herein show that the DEC205-J5 fusion significantly reduced severity of EAE symptoms compared to other treatments, and that the effective dose of the fusion protein was surprisingly lower than that of control treatments.

FIG. 2 panel A shows data obtained from administration of compositions herein to SJL mice induced for EAE. Appearance of clinical signs of EAE was monitored daily. Ten days before immunization, mice were injected introperitoneally (i.p.) with each of: anti-DEC205-J5 fusion (1 μg; closed diamonds), J5 (300 μg; asterisks; SEQ ID NO: 1), and control (none; closed circles). Ten days later (day 0) all mice were injected subcutaneously (s.c.) with 75 μg PLP139-151 in CFA, followed by pertussis toxin (PT; 200 ng intravenously; i.v.) at day 1.

FIG. 2 panel B shows data similar to that in FIG. 2 panel A, that compares data from a group of mice administered anti-DEC205-J5 fusion with that of a group of mice administered an control antibody fusion. Ten days before immunization, mice were injected i.p. with each of: anti-DEC205-J5 fusion (1 μg; closed diamonds) and control GL117-J5 fusion (1 μg; closed triangles). GL117 is a bacterial anti-β-galactosidase nonspecific isotype-matched rat monoclonal antibody negative control (Hawiger, D. et al. 2001 J Exp Med 194: 769). At day 0, all mice were injected s.c. with 75 μg PLP139-151 in CFA, followed by PT (200 ng; i.v.) at day 1.

FIG. 3 is a set of line graphs showing data in addition to that shown in FIG. 2 panel B. Ten days before immunization, mice were injected i.p. with each of anti-DEC205-J5 fusion (1 μg; closed triangles), control GL117-J5 fusion (1 μg; closed squares) and none (closed diamonds). This data shows that no amelioration of EAE symptoms is induced by GL117-J5 control fusion with average disease scores of 4.0-4.5 as compared to FIG. 2 panel B showing amelioration of the disease to some extent (average score 2.0-2.5).

FIG. 4 is a set of line and bar graphs showing data obtained from treatment of DCs with fusion monoclonal antibodies. Data herein show that DEC 205-PLP specific (also notated herein as αDEC205/PLP) monoclonal antibodies (mAB) incubated with CD11c⁺ DC- and PLP-specific T cells induced T-cell proliferation and a Th1 cytokine response.

FIG. 4 panel A is a set of bar graphs showing that T-cell proliferation occurred only in the presence of DEC205-PLP-specific monoclonal antibodies. Splenic PLP139-151 TCR transgenic CD4⁺ T cells herein were cocultured with CD11c⁺ DCs in the presence of 1 μg each of fusion monoclonal antibodies: DEC205-PLP-specific (notated αDEC205/PLP), GL117-PLP-specific (notated GL117/PLP) and none (control).

FIG. 4 panel B shows that CD11c⁺ DCs cocultured with PLP139-151-specific T cells proliferated only in the presence of DEC205-PLP-specific monoclonal antibodies. Naïve CD11c⁺ DCs herein were isolated from 40 SJL mice and coincubated with either 1 μg of DEC205-PLP (αDEC205/PLP)-specific monoclonal antibodies or GL117/PL-specific monoclonal antibodies in the presence of a PLP139-151-specific T-cell line.

FIG. 4 panel C shows data obtained from examining supernatants collected from day 3 cocultures for secretion of cytokines IL-2, IL-4, IL-10, and IFN-γ by cytokine bead array. Data herein show that only cytokine IFN-γ level was elevated after treatment with DEC205-PLP-specific monoclonal antibodies and not with GL117-PLP-specific monoclonal antibodies.

FIG. 5 is a set of line graphs showing that DEC205-PLP (αDEC205/PLP) specific monoclonal antibodies ameliorate EAE induced by adoptive transfer of pathogenic PLP139-151-specific T cells. PLP139-151-specific T-cell lines were generated as described in Examples herein and 5×10⁶ cells were adoptively transferred into naïve SJL/J mice i.v. into the tail veins. One day later, mice were immunized i.p. with either 1 μg of DEC205-PLP specific (closed diamonds; αDEC205/PLP; n=5) monoclonal antibodies or GL117-PLP-specific (closed squares; n=5) monoclonal antibodies and followed by injection of PT (200 ng; i.v.) on day 3. Mice were monitored for 30 days. Data show that anti-DEC205-PLP-treated mice were protected, and the anti-GL117-PLP-treated mice developed severe disease (P<0.02 at 30 days).

FIG. 6 is a set of line graphs showing effect of preadministration of DEC205-PLP-specific monoclonal antibodies to subjects on EAE disease course.

FIG. 6 panels A and B show data obtained from administration of the compositions herein to SJL/J mice. Mice were preimmunized by administering of each of 1 μg of fusion antibodies in sterile PBS: DEC205-PLP-specific (closed diamonds; αDEC205/PLP), GL117-PLP-specific (closed squares; GL117/PLP) or negative control (none; closed triangles) ten days before induction of EAE as shown on panel A or fifteen days before induction of disease as shown on panel B. To induce EAE, SJL/J mice were immunized with 75 μg of PLP139-151 in CFA s.c. on day 0 followed by 200 ng if PT i.v. on day 1. Appearance of clinical signs of EAE was monitored daily, and disease severity was scored as described in Examples herein. Mean EAE scores for 5-10 mice in each group are shown. The majority of mice in groups immunized with PLP139-151 that had received control fusion antibodies each of: DEC205-specific (not shown here), DEC205-HA-specific (not shown here), or GL117-PLP specific (closed diamonds) were dead by day 12 and disease was ameliorated in those that received anti-DEC205-PLP (closed diamonds). Panel A herein shows amelioration of EAE symptoms on day 15 after induction of the disease in subjects treated with anti-DEC205-PLP monoclonal antibodies (n=5) as compared to subjects treated with anti-GL117-PLP monoclonal antibodies (n=5; P<0.01). Panel B shows amelioration of EAE symptoms on day 15 in subjects treated with anti-DEC205-PLP monoclonal antibodies (n=13) compared to subjects treated with anti-GL117-PLP monoclonal antibodies (n=9; P<0.001). All scoring was performed double blind. The data shown are representative of three to six separate experiments.

FIG. 6 panel C shows effect of preimmunization with fusion monoclonal antibodies together with an adjuvant monophosphoryl lipid A (MPLA). 10 μg of MPLA was administered together with either anti-DEC205-PLP monoclonal antibodies (n=8) or anti-GL117-PLP monoclonal antibodies (n=5) i.p. ten days before induction of EAE with PLP139-151 in CFA s.c. and 200 ng of PT i.v. as above. Mice that received MPLA+anti-DEC205-PLP monoclonal antibodies were not significantly different from controls (P>0.05). A representative of two independent experiments is shown. All scoring was performed double blind.

FIG. 7 is a set of line graphs, bar graphs and photographs showing effect of preimmunization with DEC205-PLP-specific monoclonal antibodies on splenocyte proliferation and number of IL-17-producing cells.

FIG. 7 panel A is a set of line graphs showing splenocyte proliferation response. SJL/J mice herein were preimmunized with each of 1 μg of fusion antibodies: DEC205-PLP-specific (closed diamonds; αDEC205/PLP mAb) or GL117-PLP-specific (closed squares; GL117/PLP mAb). After ten days, mice were immunized with PLP139-151 (closed triangles) followed by i.v. PT as described in FIG. 5. Seventeen days after disease induction, splenocytes were removed and challenged with a titration of PLP139-151. On day 4 of the proliferation assay, cells were pulsed with ³[H]-thymidine; 16 hours later, proliferative response was measured as cpm.

FIG. 7 panel B is a set of photographs showing data of ELISPOT analysis. The data shows the number of pathogenic IL-17-secreting cells in splenocytes from SJL mice that were either left untreated or pretreated with a single injection of each of 1 μg of fusion antibodies: DEC205-PLP-specific (αDEC205/PLP mAb), control GL117-PLP-specific (GL117/PLP mAb) or control DEC205-HA (hemmaglutinin; αDEC205/HA mAb) followed by PLP139-151/CFA/PT immunization ten days later. IL-17 ELISPOT analysis was performed on mouse splenocytes isolated on day 17. Splenocytes were plated onto precoated plates as described in protocols from eBioscience's IL-17 ELISPOT kit and stimulated with 10 μg/mL PLP139-151. Unstimulated wells were used as controls. A representative of two independent experiments is shown.

FIG. 7 panel C is a set of bar graphs showing data from quantification of IL-17 ELISPOT. Statistical analysis shows that treatment with anti-DEC205-PLP monoclonal antibodies resulted in significant reduction in the number of cells secreting IL-17 compared with mice that were not pretreated (P<0.02), were pretreated with GL117-PLP monoclonal antibodies (P<0.006) or were pretreated with DEC205-HA monoclonal antibodies (hemmaglutinin; αDEC205/HA; P<0.03). Spots per million were calculated by multiplying the average of triplicate wells (2×10⁵ cells) by 5-fold.

FIG. 8 is a set of line graphs showing that adoptive transfer (ATx) of CD4⁺ T cells from mice preimmunized with DEC205/PLP139-151-specific monoclonal antibodies ameliorates induction of PLP139-151-induced EAE. Data from two independent experiments are shown at panels A and B.

FIG. 8 panel A shows effect of adoptive transfer of CD4⁺ T cells from mice pretreated with DEC205-PLP-specific monoclonal antibodies. SJL mice were preimmunized on day 10 i.p. with each of 1 μg fusion monoclonal antibodies: DEC205-PLP-specific (closed diamonds; αDEC205/PLP mAb CD4⁺ T cells) or control GL117-PLP-specific (closed squares; GL117/PLP mAb CD4⁺ T cells), or control treatment with 500 μg of the synthetic amino acid copolymer (poly (F,Y,A,K)n; closed light gray circles; PLP139-151+FYAK CD4⁺ T cells) or no treatment (closed triangles). The 5×10⁶ CD4+ T cells, enriched splenocytes obtained from preimmunized mice, were adoptively transferred i.v. into the tail veins of non-treated mice, followed by immunization on day 1 with 75 μg of PLP139-151 in CFA and PT i.v. the following day. Controls received PBS injections. Mean disease scores of five mice/group are shown. At days 20-21, data from adoptive transfer of anti-DEC205-PLP monoclonal antibodies was compared to that from adoptive transfer of anti-GL117-PLP monoclonal antibodies (P<0.02).

FIG. 8 panel B shows additional data. SJL mice herein were preimmunized at day 10 i.p. with 1 μg of DEC205-PLP specific monoclonal antibodies (closed diamonds; αDEC205/PLP mAb) only or not treated (closed triangles) in a control group. Mice were monitored for clinical signs of EAE for 30 days. All scoring was performed double blind.

FIG. 9 is a set of line and bar graphs and fluorometric dot plots showing effect of treatment of adoptively transferred CD4⁺ Vβ6⁺ TCR 5B6 transgenic (tg) T cells with DEC205-PLP-specific monoclonal antibodies. Splenocytes herein were isolated from B10.S mice that carry a transgenic TCR 5B6 recognizing PLP139-151 presented on I-A⁵. Splenocytes were enriched for Vβ6⁺ CD4⁺ tg T cells using Miltenyi CD4-positive selection kits (about 89% purity).

FIG. 9 panels A and B is a set of line graphs showing proliferation of T cells. The 10×10⁶ T cells were injected i.v. into naïve B10.S rag^{-/-} mice along with each of 1 μg i.p. of fusion antibodies: DEC205-PLP-specific (closed light gray triangles; αDEC205/PLP mAb) or GL117-PLP-specific (black asterisks; GL117/PLP mAb). Splenocytes (SP) as shown on panel A and axillary lymph nodes (LN) as shown on panel B were removed ten days later Single cell suspensions were stimulated with PLP139-151 for four days, and ³H-thymidine incorporation was measured. The data show that Vβ6⁺ CD4⁺ tg T cells treated with DEC205-PLP-specific monoclonal antibodies did not proliferate in response to PLP139-151 peptide, and Vβ6⁺ CD4⁺ tg T cells treated with GL117-PLP-specific antibodies proliferated (P<0.03).

FIG. 9 panel C is a set of bar graphs showing concentration of cytokines IL-4, IL-6, IL-10, IL-17, IFN-γ and TGFβ1 in supernatants collected from the cell proliferation assay. Vβ6⁺ TCR 5B6 tg CD4⁺ T cells herein were stimulated by cross-linking using plate-bound CD3 and CD28 monoclonal antibodies coated overnight to detect cytokine production. Supernatants from the proliferation assay were removed three days after stimulation, and cytokines were measured by Luminex assay as described in Examples herein. The data show that concentration of the cytokine IL-17 was significantly reduced upon administration of 1 μg of DEC205-PLP-specific monoclonal antibodies (white bar) compared with a control group treated with 1 μg of GL117-PLP-specific antibodies (black bar; P<0.005). The data show low concentration of IL-17 in supernatants of negative controls upon administration of αDEC205/PLP mAb (light gray bar) and GL117/PLP mAb (lighter shade of gray bar).

FIG. 9 panel D shows data obtained from fluorescence-activated cell-sorting (FACS) analysis of gated CD4⁺ cells stained for intracellular Foxp3 using markers CD4-FITC and Foxp3-PE. Splenocytes used were obtained in treatment shown in panel A. The data show 15% of Foxp3⁺ cells among CD4⁺ cells in mice pretreated with each of DEC205/PLP-specific and GL117/PLP-specific antibodies.

### DETAILED DESCRIPTION

The present invention in embodiments provides fusion proteins that bind to a dendritic cell receptor such as DEC205 and have amino acid sequences of an antibody protein specific for that receptor, fused to amino acid sequences of an immunosuppressive or a tolerogenic peptide such as J5 peptide or self-peptide proteolipid protein (PLP) 139-151.

J5 peptide is a 15mer (Stern, J. N. et al. 2005 Proc Natl Aca Sci USA 102(5):1620; Strominger, J. et al., U.S. Pat. No. 6,930,168 issued Aug. 16, 2005) that induces proliferation of IL-10 secreting regulatory T cells in mice, a property that is useful for treatment of multiple sclerosis and other autoimmune diseases. However, effective therapeutic amounts are very large. A related issue is that peptide compositions are readily hydrolyzed in vivo.

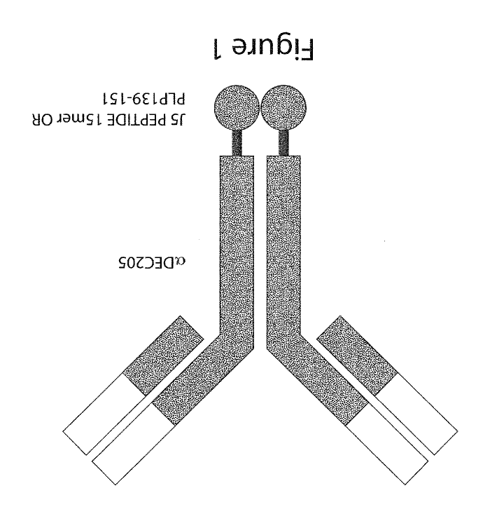

FIG. 1 shows a drawing of the structure of anti-DEC205 antibody fused to the immunosuppressive J5 peptide or the tolerogenic PLP139-151 peptide that targets the DEC205 receptor. The peptide herein is covalently bound to the carboxy terminus of the H chains of the IgG antibody. The peptides shown in FIG. 1 are exemplary only and not further limiting.

In certain embodiments of the invention, anti-DEC205-mediated delivery of the tolerogenic self-peptide proteolipid protein (PLP) 139-151 to DCs ameliorated clinical symptoms in the PLP-induced SJL model of experimental autoimmune encephalomyelitis. Splenocytes from treated mice were anergized to PLP139-151, and IL-17 secretion was markedly reduced. Examples herein show directly, using transgenic CD4⁺ Vβ6⁺ TCR T cells specific for PLP139-151, that under the conditions of the present examples, these cells also became anergic. In addition, evidence for a CD4⁺ T cell-mediated suppressor mechanism was obtained.

The invention herein provides a novel therapy for demyelinating diseases by inducing in vivo expansion of regulatory T cells by targeting the immunosuppressive peptide J5 directly to dendritic cells in order to generate immunosuppressive IL-10-secreting T cells at high frequency.

The random amino acid copolymer Copaxone [poly(Y, E, A, K)ₙ], termed YEAK, is a primary therapy for relapsing, remitting multiple sclerosis. Poly(F, Y, A, K)ₙ, termed FYAK, a second generation Copaxone, was developed based on the structure of HLA-DR2 (DRB1*1501/DRA), the MHC protein to which MS is linked and to which these copolymers bind (Wucherpfennig, K. W. et al. 1994 J Exp Med 179:279; Kalandadze, A. et al. 1996 J Biol Chem 271: 20156; Gauthier, L. et al. 1998 Proc Natl Acad Sci 95: 11828; Smith, K. J. et al. 1998 J Exp Med 188: 1511). Particularly important was the absence in YEAK of an amino acid whose side chain would provide high affinity for the important P1 pocket of HLA-DR2. FYAK, the F residue of which fits tightly into the P1 pocket, is far more effective than Copaxone in the treatment of Experimental Autoimmune Encephalomyelitis (EAE, the mouse model of MS) (Strominger, J. L. 2002 J Clin Invest 109: 1635; Stern, J. N. et al. 2004 Proc Natl Acad Sci 101: 11743; Illes, Z. et al. 2004 Proc Nall Acad Sci 101: 11749), has completed a Phase Ib clinical trial (Kovalchin, J. et al. 2010 J Neuroimmunol Epub ahead of print May 11, 2010), and will soon begin a Phase II clinical trial. The 15-mer peptide J5, a third generation material, was developed based on the motif for binding of Copaxone to HLA-DR2, and is also far more effective than Copaxone in the treatment of EAE (Fridkis-Hareli, M. et al. 2001 Hum Immunol 62: 753; Stern, J. N. et al. 2005 Proc Natl Acad Sci 102: 1620) All three of these materials function by inducing the generation of IL-10 secreting regulatory T cells and also by affecting changes in the antigen presenting cell, macrophages (Stern, J. N. et al. 2008 Proc Natl Acad Sci 105: 5172; Illes, Z. et al. 2005 Eur J Immunol 35: 3683; Weber, M. S. et al. 2007 Nature Medicine 13: 935; Hong, Z., et al. 2009 Proc Natl Acad Sci 106(9): 3336). J5 is the most potent of these immunosuppressive agents, but like other peptides its use in therapies is limited by its distribution throughout the body and degradation in serum. Multiple sclerosis patients have been shown to have a dysfunction and/or deficit of regulatory T cells (Costantino, C. M. et al. 2008 J Clin Immunol 28: 697; Allan, S. E. et al. 2008 Immunol Rev. 223:391), and, thus, the in vivo generation of these T cells would result in replacement of the deficient function.

The hypothesis is that fusion complexes of the monoclonal antibody DEC205 and/or 33D1 located on distinct sets of dendritic cells with the immunosuppressive peptide J5 will provide a powerful way to induce tolerance to induction of EAE in mice. Exceedingly small amounts of the fusion complexes will suffice to induce tolerance and will terminate relapses in the relapsing, remitting model of EAE. These studies would provide the basis for developing a new therapy for the treatment of relapsing, remitting MS and to protect patients from subsequent relapses of the disease. The lack or dysfunction of regulatory T cells has been described in MS patients as well as in patients with other autoimmune diseases (Costantino, C. M. et al. 2008 J Clin Immunol 28: 697; Allan, S. E. et al. 2008 Immunol Rev. 223: 391). The proposed therapy would aim to increase the pool of regulatory T cells in mice and, later, in MS patients.

FYAK is being developed for clinical use by Peptimmune, Inc., which licensed the patent from Harvard. FYAK continues to hold promise as a more effective substitute for Copaxone. In addition it is effective when administered weekly s.c. at the low doses of 3 or 10 mg. (in contrast to Copaxone which is administered daily s.c. at a dose of 20 mg, i.e., 140 mg/week). A Phase Ib study of 50 patients with secondary progressive MS revealed its ability given weekly at these doses to severely reduce the number of Gadolinium-enhancing lesions and to induce the presence in serum of the same cytokines as had been found in studies of EAE (Kovalchin, J. et al. 2010 J Neuroimmunol Epub ahead of print May 11). The Phase II study will be composed of 350 patients with relapsing remitting MS and will extend over 1.5 years. Importantly, the anti-DEC205-J5 fusion protein may be an even more potent drug with the same effects but requiring even smaller amounts and at a lower frequency. Administration weekly or possibly at even lower frequency is important because the pain associated with daily administration of Copaxone results in discontinuance of this therapy by some MS patients and limits the frequency with which it is prescribed.

Self-reactive T cells with known antigen specificity, which can be found in T cell-mediated autoimmune diseases such as multiple sclerosis, are used herein as targets for antigen-specific tolerance induction without compromising host immunity to infectious insults. Various protocols have been used to interfere with unwanted immunity using peptide-induced tolerance (Miller, S. D. et al. 2007 Nat Rev Immunol 7-665), including the administration of antigens over extended periods of time using osmotic minipumps (Verginis, P. et al. 2008 Proc Natl Acad Sci USA 105:3479; Apostolou, I. et al. 2004 J Exp Med 199:1401).

In addition, peptide antigens are directly delivered to antigen-presenting cells using targeting approaches. In particular, antigens delivered to different subsets of DCs after fusion with antibodies to the endocytic receptors DEC205 (anti-DEC205) or 33D1 are efficiently processed and presented by MHC class I and class II molecules (Dudziak, D. et al. 2007 Science 315:107). This route of antigen delivery to murine (Hawiger, D. et al. 2001 J Exp Med 194:769) or human (Bozzacco, L. et al. 2007 Proc Nall Acad Sci USA 104:1289) DCs is several orders of magnitude more efficient than free peptides and in conjunction with maturation stimuli represents an effective method for inducing strong T-cell responses, i.e., vaccination. By contrast, targeting antigen to immature DCs in the steady state has been described as promoting immunological tolerance through different mechanisms in different studies (Hawiger, D. et al. 2001 J Exp Med 194:769, Hawiger, D. et al. 2004 Immunity 20:6955; Kretschmer, K. et al. 2005 Nat Immunol 6:1219; Mukhopadhaya, A. et al. 2008 Proc Nall Acad Sci USA 105:6374; Bruder, D. et al. 2005 Diabetes 54:3395). It leads to deletion of antigen-specific T cells with residual cells becoming immunologically unresponsive, a mechanism that in one study increased CD5 expression on activated T cells (Hawiger, D. et al. 2004 Immunity 20:695). In addition, delivering minute amounts of peptides using anti-DEC205 fusion proteins to steady-state immature DCs leads to the de novo generation of antigen-specific Foxp3⁺ Treg in vivo (Kretschmer, K. et al. 2005 Nat Immunol 6:1219; Yamazaki, S. et al. 2008 J Immunol 181:6923).

Anti-DEC205-mediated targeting of an encephalogenic peptide of the myelin oligodendrocyte glycoprotein (MOG), a minor myelin component, to DCs in vivo prevents EAE induction by subsequent injection of the same peptide in complete Freund's adjuvant (CFA) in C57BL/6 mice (Hawiger, D. et al. 2004 Immunity 20:695). In this model, pretreatment with large doses of the free peptide in the absence of adjuvants also leads to protection from subsequent challenge. Examples herein show anti-DEC205-mediated targeting of the tolerogenic autoantigen of the proteolipid protein peptide (PLP139-151) (derived from a major myelin constituent) in the EAE model in SJL mice, which is much more prone to disease and in which free peptide administration does not lead to protection. This model represents a second example in which targeting of DCs in the steady state with nanogram amounts of a peptide that generates autoimmunity efficiently ameliorates disease by promoting tolerance. In the present case, the amelioration of disease results both from induction of T-cell anergy and by generation of suppressor T cells.

DCs are specialized cells of the immune system that play a major role in distinguishing immunologically exogenous antigens from the self DCs function centrally in thymus, and in the periphery (Steinman, R. et al. 2003 Ann N Y Acad Sci 987:15). DCs have the capacity for initiating primary and secondary T and B lymphocyte responses by presenting antigens in the form of peptides bound to cell-surface major histocompatibility complex (MHC) molecules.

Additionally, immature dendritic cells in the steady state were found to be tolerogenic in several experimental models. For example, administration of peptides that can induce EAE to tolerogenic DCs (immature steady state DCs) resulted in tolerance to autoimmune disease, rather than in its stimulation (Hawiger, D. et al. 2001 J Exp Med 194:769; Mahnke, K. et al. 2003 Blood 101:4862). Dendritic cells express a variety of surface receptors that function to endocytose antigens that bind to them for subsequent degradation and presentation as peptides to the immune system. Different subsets of DC have been described that degrade protein antigens in different ways and present them on either MHC Class I or Class II proteins (Dudziak, D. et al. 2007 Science 315: 107).

One subset, CD8α DC, utilizes the lectin DEC205 for endocytosis while another subset, CD4 DC, utilizes the lectin 33D1 for endocytosis. A technology was developed to link pathogenic peptides to the C-terminus of a monoclonal antibody directed against DEC205 (anti-DEC205 or αDEC205; Hawiger, D. et al. 2001 J Exp Med 194: 769). Upon binding to DEC205, the αDEC205-peptide fusion complex is endocytosed. The fused peptide is released by proteolysis and, when administered to immature DC, tolerance to this peptide is induced. Several studies with immunostimulatory peptides that produce EAE when injected under appropriate conditions have been used to construct anti-DEC205-peptide fusion complexes (Hawiger D. 2004 Immunity 20: 695; Kretschmer, K. et al. 2006 Nature Immunol 6: 1219; Stem, J. N. H. et al. 2010 Proc Natl Acad Sci published on line). In all cases the pathogenic peptide in the fusion complex induced tolerance to subsequent attempts to elicit EAE (or diabetes; Bruder, D., et al. 2005 Diabetes 54: 3395) when administered as the anti-DEC205-peptide fusion complex. Extremely small amounts were effective, i.e., 1 μg of fusion complex equivalent to about 20 ng of peptide. No studies have been published that utilize anti-33D1 fusion complexes to target the CD4+ DC subset. Neither have there been studies of targeting immunosuppressive peptides, rather than immune activating peptides, to DC.

Several DC receptors have been identified, for example, DCs display mannose receptors (MR) and use MR-mediated endocytosis for efficient antigen capture and targeting to the endosomal/lysosomal compartment. Types of MR expressed by human dendritic cells include high homology to the DEC205 found in mice; and high homology to that expressed in human macrophages (Kato, M. et al. 1998 Immunogenetics 47:442; Ezekowitz, R. A. et al. 1990 J Exp Med 172:1785). While examples herein use DEC205, without being limited by any particular theory or mechanism of action any of the receptors on a dendritic cell are here envisioned as suitable targets for a composition that is a fusion of a peptide herein and an antibody protein that specifically binds that dendritic cells receptor.

A type of dendritic cell (DC1) expresses Toll-like receptors (TLR). Upon binding natural ligands, for example, pathogens, by these receptors, DC1 become activated and mature into antigen-presenting cells that secrete Th-1 or Th-2 cytokines and prime naïve T cells for a proper immune response. Exemplary TLRs identified in humans and mice, respectively, are described by Tabeta, K. et al. 2004 Proc Natl Acad Sci USA 101:3516 and Kawai, T. et al. 2005 Curr Opin Immunol 17:338.

Dendritic cell receptor CLEC9A couples sensing of necrosis to immune responses mediated by cells, functioning as a tyrosine-kinase SYK-coupled C-type lectin receptor regulating cross-priming to cell-associated antigens, and thus mediating sensing of necrosis by the dendritic-cells (Sancho, D. et al. 2009 Nature 458:899).

DEC205 is an endocytic receptor expressed at high level in DCs. Monoclonal antibodies specific to DEC205 are well known (Kraal, G. et al. 1986 J Exp Med 163:98; Witmer-Pack, M. D. et al. 1995 Cell Immunol 163:15). Anti-DEC205 antibody compositions include NLDC-145, which is a nonlymphoid DC product of 145 kDa. The target recognized by NLDC-145 is expressed by DCs, including nonlymphoid DCs, e.g. Langerhans cells of epidermis, and by DCs in lymphoid tissues, such as the thymic cortical epithelium. The purified and cloned DEC205 target has a molecular weight of 205 kDa, with 10 contiguous C-type lectin domain, a decalectin of molecular weight 205 kDa. Anti-DEC205 monoclonal antibody, NLDC-145, is available commercially (Imgenex, San-Diego, Calif., CD205, DEC205, clone NLDC-145 Cat. No. DDX0020A488; Cell Sciences, Canton, Mass., mouse CD205, DEC205, clone NLDC-145, Cat. No. HM1069; AbD Serotex, Raleigh, N.C., anti mouse CD205, DEC205, clone NLDC-145, Cat. No. MCA949; Bachem Chemicals, Torrance, Calif., DEC205, clone-145, Cat. Nos. T-2013, T-2025, T-2023; U.S. patent publication serial number 2009/017588 published Jul. 9, 2009, shows anti-DEC205 monoclonal antibodies). Tissue distribution of DEC205 protein in lymphoid and non-lymphoid tissue was shown using NLDC-145 anti-DEC205 monoclonal antibody (Witmer-Pack, M. D. et al. 1995 Cell Immunol 163:15). DEC205 receptor had been characterized extensively (Jiang, W. et al. 1995 Nature 375: 151; Mahnke, K. et al. 2000 J Cell Biol 151:673). A fusion protein cDNA combining the extracellular portion of DEC205 with a constant region of immunoglobulin was sequenced (GENBANK Accession number ABD72617).

Studies of multiple sclerosis are facilitated by the animal model experimental autoimmune encephalomyelitis (EAE) that recapitulates many aspects of the human disease (Wekerle, H.1993 Curr Opin Neurobiol 3:779). Active induction of EAE is accomplished by stimulation of T cell-mediated immunity to myelin, the insulating phospholipid layer surrounding the neuronal axons, through immunization with myelin proteins or synthetic peptide antigens derived from myelin and then emulsified in adjuvant (Tuohy, V. K. et al. 1989 J Immunol 142:1523). This treatment leads to activation of autoreactive myelin-specific CD4⁺ T cells that circulate in the periphery of experimental animals. Activated autoreactive T cells will cross the blood-brain barrier (Risau, W. et al. 1990 J Cell Biol 110:1757). Within the central nervous system, local and infiltrating antigen-presenting cells, such as dendritic cells (DCs) derived from microglia, present MHC class II molecule-associated myelin peptides to infiltrating T cells in the context of costimulation. Myelin-specific CD4⁺ T cells are reactivated, initiating a cascade of neuroinflammatory responses that ultimately leads to demyelination in the central nervous system and neurodegeneration. EAE can also be passively induced by adoptive transfer of pre-activated myelin-specific T cells (Stromnes, I. M. et al. 2006 Nat Protoc 1:1810).

Although T helper 1 (Th1) cells secreting IFN-γ were considered to be the primary mediators of EAE, T helper 17 (Th17) cells also were shown to exhibit greater pathogenicity, suggesting that they play a more decisive role in mediating severe tissue damage (McKenzie, B. S. et al. 2006 Trends Immunol 27:17; Bettelli, E. et al. 2007 Nat Immunol 8:345). However, both Th1 and Th17 cells, generated with kinetic differences and/or involved at different stages, may be involved in development of EAE (Steinman, L. 2007 Nat Med 13:139). In fact, the relative contribution of both Th subsets affects the anatomical location of lesion distribution between brain and spinal cord parenchyma (Stromnes, I. M. et al. 2008 Nat Med 14:337).

### Targeting Dendritic Cells.

Approach of invention herein is to target the immunosuppressive peptide J5 directly to dendritic cells (DCs) in mice in order to generate IL-10-secreting regulatory T cells at high frequency using an anti-DEC205-J5 fusion protein and an anti-33D1-J5 fusion protein as the targeting agents and to examine the generation of these regulatory T cells in vivo and their effect on susceptibility to EAE.

An example herein shows anti-DEC205-PLP139-151 complex and described in Stern, J. N. H. et al. 2010 Proc Natl Acad Sci, published on line, incorporated herein by reference hereby in its entirety. PLP139-151 is a peptide that induces EAE in SJL mice. Phospholipoprotein (PLP) is also involved as an autoantigen in multiple sclerosis (Zhang, J. et al. 1994 JEM 179: 3973). Anti-DEC205-mediated delivery of PLP139-151 to DC using 1 μg of the fusion complex ameliorated clinical symptoms in the PLP-induced model of EAE. T cells in splenocytes from treated mice were anergized to PLP139-151 and IL-17 secretion was markedly reduced. In addition, evidence for a CD4+ T cell-mediated suppressor mechanism was obtained. Moreover, transgenic CD4+Vβ6+5B6 TCR T cells specific for PLP139-151 (Waldner H. et al. 2000 Proc Natl Acad Sci 97: 3412) adoptively transferred i.v. into rag mice were shown to become anergic after treatment with the anti-DEC205-PLP139-151 fusion complex and were not deleted (Stern, J. N. H. et al. 2010 Proc Natl Acad Sci, published on line).

### Duration.

Most important is the duration of the vaccination effect with αDEC205-J5. Thus, SJL mice herein are vaccinated 10, 14, and 21 days prior to immunization with PLP139-151. Longer prevaccination periods are also examined.

### Dose.

A second important parameter is the dose of anti-DEC205-J5 employed. Thus, doses of 0.1, 0.3, 1, 3, and 10 μg are examined. Since the duration of the effect may be related to the dose used, an additional experiment examines whether the duration can be prolonged with a larger dose. These experiments are important because a primary obstacle to the present use of Copaxone for the treatment of MS is patient compliance given pain associated with daily injections. Thus, the amount injected using anti-DEC205-J5 is considerably smaller (in the mouse about 50-100 μg of Copaxone compared to 1 μg anti-DEC205-J5 complex; about 20 ng of J5), and administration is every two to three weeks rather than daily. Data have also been obtained indicating that alum as an adjuvant is very effective when administered with the amino acid copolymers in reducing the dosage required (Tartaglia, C. 2009 Suppression of a mouse model of multiple sclerosis by immunization with an aluminum adjuvant and low dosages of copolymer FYAK, Undergraduate Honors Thesis in Biochemical Sciences, Harvard College).

Its potentiating effect on administration of J5 and particularly of anti-DEC205-J5 is therefore also examined.

### Mechanism.

The mechanism of protection is generation of IL-10 secreting regulatory T cells as it is for the amino acid copolymers and for the J5 peptide 15mer itself, as confirmed by studying induction of proliferation of splenic T cells by the anti-DEC205-J5 fusion complex and the level of IL-10 secretion by these T cells.

### Targeting to Immature or Mature DC.

Examples herein answer the question of whether immature or mature dendritic cells were most important in generating protection using anti-DEC205-PLP139-151 (Stern, J. N. H. et al. 2010 Proc Natl Acad Sci, published on line). Mice were used without prior treatment (steady state immature dendritic cells) or in which mice were first treated with 10 μg monophosphoryl lipid A (MPLA), a nontoxic derivative of Lipid A that induces maturation of DC (Mata-Haro, V. et al. 2007 Science 316: 1628). Anti-DEC205-PLP139-151 was effective only when administered to mice in the steady state. This result shows that DC could function in tolerization to foreign antigens only when steady state immature DC were used. It is not clear whether treatment with an immunosuppressive peptide such as J5 would also require immature DC or whether mature DC might be more effective in this type of vaccination. Experiments therefore are carried out in which mice are pretreated with 10 μg of MPLA prior to vaccination with anti-DEC205-J5 to show which type of DC functions best with this immunosuppressive peptide.

An aspect of the invention provides a composition including a fusion protein having an amino acid sequence of a monoclonal antibody specific for binding to a dendritic cell receptor protein (e.g. DEC205; GENBANK Accession number AAC17636A) and an amino acid sequence of peptide EKPKVEAYKAAAAPA (SEQ ID NO: 1). Examples of additional dendritic cell receptors include mannose receptors, Toll-like receptors, C-type lectin receptors, such as 33D1 and CLEC9A. For example, the peptide has an amino acid sequence selected from the group: EKPK (SEQ ID NO: 2); KPKV (SEQ ID NO: 3); PKVE (SEQ ID NO: 4); KVEA (SEQ ID NO: 5); VEAY (SEQ ID NO: 6); EAYK (SEQ ID NO: 7); AYKA (SEQ ID NO: 8); YKAA (SEQ ID NO: 9); KAAA (SEQ ID NO: 10); AAAA (SEQ ID NO: 11); AAAP (SEQ ID NO: 12); AAPA (SEQ ID NO: 13); EKPKV (SEQ ID NO: 14); KPKVE (SEQ ID NO: 15); PKVEA (SEQ ID NO: 16); KVEAY (SEQ ID NO: 17); VEAYK (SEQ ID NO: 18); EAYKA (SEQ ID NO: 19); AYKAA (SEQ ID NO: 20); YKAAA (SEQ ID NO: 21); KAAAA (SEQ ID NO: 22); AAAAP (SEQ ID NO: 23); AAAPA (SEQ ID NO: 24); EKPKVE (SEQ ID NO: 25); KPKVEA (SEQ ID NO: 26); PKVEAY (SEQ ID NO: 27); KVEAYK (SEQ ID NO: 28); VEAYKA (SEQ ID NO: 29); EAYKAA (SEQ ID NO: 30); AYKAAA (SEQ ID NO: 31); YKAAAA (SEQ ID NO: 32); KAAAAP (SEQ ID NO: 33); AAAAPA (SEQ ID NO: 34); EKPKVEA (SEQ ID NO: 35); KPKVEAY (SEQ ID NO: 36); PKVEAYK (SEQ ID NO: 37); KVEAYKA (SEQ ID NO: 38); VEAYKAA (SEQ ID NO: 39); EAYKAAA (SEQ ID NO: 40); AYKAAAA (SEQ ID NO: 41); YKAAAAP (SEQ ID NO: 42); KAAAAPA (SEQ ID NO: 43); EKPKVEAY (SEQ ID NO: 44); KPKVEAYK (SEQ ID NO: 45); PKVEAYKA (SEQ ID NO: 46); KVEAYKAA (SEQ ID NO: 47); VEAYKAAA (SEQ ID NO: 48); EAYKAAAA (SEQ ID NO: 49); AYKAAAAP (SEQ ID NO: 50); YKAAAAPA (SEQ ID NO: 51); EKPKVEAYK (SEQ ID NO: 52); KPKVEAYKA (SEQ ID NO: 53); PKVEAYKAA (SEQ ID NO: 54); KVEAYKAAA (SEQ ID NO: 55); VEAYKAAAA (SEQ ID NO: 56); EAYKAAAAP (SEQ ID NO: 57); AYKAAAAPA (SEQ ID NO: 58); EKPKVEAYKA (SEQ ID NO: 59); KPKVEAYKAA (SEQ ID NO: 60); PKVEAYKAAA (SEQ ID NO: 61); KVEAYKAAAA (SEQ ID NO: 62); VEAYKAAAAP (SEQ ID NO: 63); EAYKAAAAPA (SEQ ID NO: 64); EKPKVEAYKAA (SEQ ID NO: 65); KPKVEAYKAAA (SEQ ID NO: 66); PKVEAYKAAAA (SEQ ID NO: 67); KVEAYKAAAAP (SEQ ID NO: 68); VEAYKAAAAPA (SEQ ID NO: 69); EKPKVEAYKAAA (SEQ ID NO: 70); KPKVEAYKAAAA (SEQ ID NO: 71); PKVEAYKAAAAP (SEQ ID NO: 72); KVEAYKAAAAPA (SEQ ID NO: 73); EKPKVEAYKAAAA (SEQ ID NO: 74); KPKVEAYKAAAAP (SEQ ID NO: 75); PKVEAYKAAAAPA (SEQ ID NO: 76); EKPKVEAYKAAAAP (SEQ ID NO: 77); and KPKVEAYKAAAAPA (SEQ ID NO: 78).

The peptides in various embodiments have a length of at least 4 amino acid residues. Alternatively, the peptides have a length of at least 15 amino acid residues. The compositions can include a pharmaceutically acceptable salt, carrier or buffer.

The composition fusion protein is specific for binding to the dendritic cell receptor protein including DEC205 receptor. For example, the fusion protein has an immunomodulatory function. In certain embodiments, the immunomodulatory function is a tolerogenic or an immunosuppressive function. Further, the immunomodulatory function involves inhibition of an immune function, for example, inhibition of an MHC class II interaction, such as with T cells.

The composition includes a unit dose effective for treatment of a subject for an autoimmune condition. In general, the autoimmune condition is a demyelinating condition. The demyelinating condition in certain embodiments is multiple sclerosis (MS). In various embodiments, the autoimmune condition is a cell mediated disease, for example, is mediated by a T cell or a natural killer (NK) cell, or is an antibody mediated disease. For example, the autoimmune condition is selected from the group consisting of autoimmune hemolytic anemia, autoimmune oophoritis, autoimmune thyroiditis, autoimmune uveoretinitis, Crohn's disease, chronic immune thrombocytopenic purpura, colitis, contact sensitivity disease, diabetes mellitus, Graves disease, Guillain-Barre's syndrome, Hashimoto's disease, idiopathic myxedema, multiple sclerosis, myasthenia gravis, psoriasis, pemphigus vulgaris, rheumatoid arthritis, and systemic lupus erythematosus.

The present invention also features a kit for treating a subject having an autoimmune disease including a fusion of amino acid sequence. Further, the kit in various embodiments includes a pharmaceutically acceptable buffer, container and instructions for use.

An aspect of the present invention provides a method for treating a subject of an autoimmune disease involving constructing a fusion protein having an amino acid sequence of a monoclonal antibody that specifically binds a dendritic cell receptor protein (e.g. GENBANK Accession number AAC17636A) and a peptide amino acid sequence such as EKPKVEAYKAAAAPA (SEQ ID NO: 1); a contacting the subject with a composition including the fusion protein; and observing reducing or eliminating symptoms of the autoimmune disease.

The method in further embodiments includes chemically linking the monoclonal antibody and the peptide. The method in various embodiments has the peptide including the amino acid sequence selected from the group of: EKPK (SEQ ID NO: 2); KPKV (SEQ ID NO: 3); PKVE (SEQ ID NO: 4); KVEA (SEQ ID NO: 5); VEAY (SEQ ID NO: 6); EAYK (SEQ ID NO: 7); AYKA (SEQ ID NO: 8); YKAA (SEQ ID NO: 9); KAAA (SEQ ID NO: 10); AAAA (SEQ ID NO: 11); AAAP (SEQ ID NO: 12); AAPA (SEQ ID NO: 13); EKPKV (SEQ ID NO: 14); KPKVE (SEQ ID NO: 15); PKVEA (SEQ ID NO: 16); KVEAY (SEQ ID NO: 17); VEAYK (SEQ ID NO: 18); EAYKA (SEQ ID NO: 19); AYKAA (SEQ ID NO: 20); YKAAA (SEQ ID NO: 21); KAA AA (SEQ ID NO: 22); AAAAP (SEQ ID NO: 23); AAAPA (SEQ ID NO: 24); EKPKVE (SEQ ID NO: 25); KPKVEA (SEQ ID NO: 26); PKVEAY (SEQ ID NO: 27); KVEAYK (SEQ ID NO: 28); VEAYKA (SEQ ID NO: 29); EAYKAA (SEQ ID NO: 30); AYKAAA (SEQ ID NO: 31); YKAAAA (SEQ ID NO: 32); KAAAAP (SEQ ID NO: 33); AAAAPA (SEQ ID NO: 34); EKPKVEA (SEQ ID NO: 35); KPKVEAY (SEQ ID NO: 36); PKVEAYK (SEQ ID NO: 37); KVEAYKA (SEQ ID NO: 38); VEAYKAA (SEQ ID NO: 39); EAYKAAA (SEQ ID NO: 40); AYKAAAA (SEQ ID NO: 41); YKAAAAP (SEQ ID NO: 42); KAAAAPA (SEQ ID NO: 43); EKPKVEAY (SEQ ID NO: 44); KPKVEAYK (SEQ ID NO: 45); PKVEAYKA (SEQ ID NO: 46); KVEAYKAA (SEQ ID NO: 47); VEAYKAAA (SEQ ID NO: 48); EAYKAAAA (SEQ ID NO: 49); AYKAAAAP (SEQ ID NO: 50); YKAAAAPA (SEQ ID NO: 51); EKPKVEAYK (SEQ ID NO: 52); KPKVEAYKA (SEQ ID NO: 53); PKVEAYKAA (SEQ ID NO: 54); KVEAYKAAA (SEQ ID NO: 55); VEAYKAAAA (SEQ ID NO: 56); EAYKAAAAP (SEQ ID NO: 57); AYKAAAAPA (SEQ ID NO: 58); EKPKVEAYKA (SEQ ID NO: 59); KPKVEAYKAA (SEQ ID NO: 60); PKVEAYKAAA (SEQ ID NO: 61); KVEAYKAAAA (SEQ ID NO: 62); VEAYKAAAAP (SEQ ID NO: 63); EAYKAAAAPA (SEQ ID NO: 64); EKPKVEAYKAA (SEQ ID NO: 65); KPKVEAYKA AA (SEQ ID NO: 66); PKVEAYKAAAA (SEQ ID NO: 67); KVEAYKAAAAP (SEQ ID NO: 68); VEAYKAAAAPA (SEQ ID NO: 69); EKPKVEAYKAAA (SEQ ID NO: 70); KPKVEAYKAAAA (SEQ ID NO: 71); PKVEAYKAAAAP (SEQ ID NO: 72); KVEAYKAAAAPA (SEQ ID NO: 73); EKPKVEAYKAAAA (SEQ ID NO: 74); KPKVEAYKAAAAP (SEQ ID NO: 75); PKVEAYKAAAAPA (SEQ ID NO: 76); EKPKVEAYKAAAAP (SEQ ID NO: 77); and KPKVEAYKAAAAPA (SEQ ID NO: 78).

Another embodiment of the method provides constructing the fusion protein by engineering a recombinant nucleic acid sequence having a nucleic acid sequence encoding a chain of the monoclonal antibody or a fragment thereof and a nucleic acid sequence encoding the peptide and expressing the recombinant nucleic acid sequence in cells. For example, the recombinant nucleic acid sequence was obtained by fusing nucleic acid sequence of the peptide to a C-terminus of a cDNA encoding a heavy chain of the antibody.

The monoclonal antibody was obtained by producing it from a hybridoma cell line.

The autoimmune disease of certain embodiments includes demyelinating condition. In alternative embodiments, the autoimmune disease is selected from: autoimmune hemolytic anemia, autoimmune oophoritis, autoimmune thyroiditis, autoimmune uveoretinitis, Crohn's disease, chronic immune thrombocytopenic purpura, colitis, contact sensitivity disease, diabetes mellitus, Graves disease, Guillain-Barre's syndrome, Hashimoto's disease, idiopathic myxedema, multiple sclerosis, myasthenia gravis, psoriasis, pemphigus vulgaris, rheumatoid arthritis, and systemic lupus erythematosus.

In general, the subject is a mammal. For example, the subject is a rodent. The rodent in further examples is a mouse with experimental allergic encephalomyelitis. Further, the rodent is a humanized mouse. The subject in an alternative embodiment is a human. For example, the human is a patient with MS.

In related embodiments, reducing is observing decreasing severity or frequency of recurrences of symptoms.

An embodiment of the method provides the composition that is administered by a route selected from the group of: intravenous (i.v.), subcutaneous (s.c), intramuscular (i.m.), and intraperitoneal (i.p.).

After contacting the subject with the composition, the method in related embodiments further involves analyzing a physiological parameter of the demyelinating condition. For example, analyzing the physiological parameter is testing T cells from the subject for reactivity to a peptide of myelin basic protein. For example, the peptide is MBP 85-99.

The method in various embodiments further includes administering an additional therapeutic agent. For example, the additional therapeutic agent is selected from the group consisting of: an antibody, an enzyme inhibitor, an antibacterial agent, an antiviral agent, a steroid, a nonsteroidal anti-inflammatory agent, an antimetabolite, a cytokine, a cytokine blocking agent, an adhesion molecule blocking agent, a soluble cytokine receptor, and a random linear amino acid copolymer composition. In further examples, the cytokine is an interferon. Further, the copolymer is selected from the group of YEAK (Copaxone®), FYAK, VWAK and VFAK.

In another embodiment the method involves an amount of the fusion protein required to induce tolerance in mice less than about 1 mg, less than about 500 μg, less than about 300 μg, or less than about 100 μg. For example, the amount is at least about 10 ng, 100 ng, 1 μg, 50 μg, 100 μg, 150 μg, 200 μg, 250 μg or 300 μg.

An embodiment provides a method for detecting the presence of DEC205 receptor in a biological sample involving contacting a biological sample with the fusion protein amino acid sequence; and detecting the fusion protein bound to the DEC205 receptor, thereby detecting DEC205 receptor.

### Monoclonal Antibodies Specific to Dendritic Cell Receptors

Antibodies specific to dendritic cell receptors such as to DEC205, are herein linked to immunosuppressive and tolerogenic peptides, to present peptide determinants that induce tolerance to T cells that recognize the determinants. Engineered heavy chains of anti-DEC205 antibody were prepared to encode a peptide from hen egg white lysozyme (HEL; Hawiger, D. et al. 2001 J Exp Med 194:769). The anti-DEC205 HEL antibody was assessed with HEL-specific and MHC II-restricted, TCR transgenic cells for presentation of HEL. Further, a hybrid anti-DEC205 antibody with a myelin oligodendrocyte glycoprotein (MOG) amino acid sequence was engineered (Hawiger et al. 2004 Immunity 20:695). Rat anti-mouse DEC205 was chemically conjugated to ovalbumin protein (OVA; Bonifaz, L. D. et al. 2002 J Exp Med 196:1627), and the antibody-OVA conjugate presentation to MHC class I and MHC class II molecules was observed. Hemagglutinin (HA) fused with anti-DEC205 antibody was observed to target influenza hemagglutinin to DCs (Kretschmer, K. et al. 2005 Nat Immun 6:1219).

The invention herein provides a fusion of an amino acid sequence of a monoclonal antibody for binding an antigen of a dendritic cell receptor protein with a tolerogenic, immunosuppressive amino acid sequence such as peptide EKPKVEAYKAAAAPA (SEQ ID NO: 1) or peptide fragments. For example, amino acid sequence is selected form the group of: EKPK (SEQ ID NO: 2), KPKV (SEQ ID NO: 3), PKVE (SEQ ID NO: 4), KVEA (SEQ ID NO: 5), VEAY (SEQ ID NO: 6), EAYK (SEQ ID NO: 7), AYKA (SEQ ID NO: 8), YKAA (SEQ ID NO: 9), KAAA (SEQ ID NO: 10), AAAA (SEQ ID NO: 11), AAAP (SEQ ID NO: 12), AAPA (SEQ ID NO: 13), EKPKV (SEQ ID NO: 14), KPKVE (SEQ ID NO: 15), PKVEA (SEQ ID NO: 16), KVEAY (SEQ ID NO: 17), VEAYK (SEQ ID NO: 18), EAYKA (SEQ ID NO: 19), AYKAA (SEQ ID NO: 20), YKAAA (SEQ ID NO: 21), KAAAA (SEQ ID NO: 22), AAAAP (SEQ ID NO: 23), AAAPA (SEQ ID NO: 24), EKPKVE (SEQ ID NO: 25), KPKVEA (SEQ ID NO: 26), PKVEAY (SEQ ID NO: 27), KVEAYK (SEQ ID NO: 28), VEAYKA (SEQ ID NO: 29), EAYKAA (SEQ ID NO: 30), AYKAAA (SEQ ID NO: 31), YKAAAA (SEQ ID NO: 32), KAAAAP (SEQ ID NO: 33), AAAAPA (SEQ ID NO: 34), EKPKVEA (SEQ ID NO: 35), KPKVEAY (SEQ ID NO: 36), PKVEAYK (SEQ ID NO: 37), KVEAYKA (SEQ ID NO: 38), VEAYKAA (SEQ ID NO: 39), EAYKAAA (SEQ ID NO: 40), AYKAAAA (SEQ ID NO: 41), YKAAAAP (SEQ ID NO: 42), KAAAAPA (SEQ ID NO: 43), EKPKVEAY (SEQ ID NO: 44), KPKVEAYK (SEQ ID NO: 45), PKVEAYKA (SEQ ID NO: 46), KVEAYKAA (SEQ ID NO: 47), VEAYKAAA (SEQ ID NO: 48), EAYKAAAA (SEQ ID NO: 49), AYKAAAAP (SEQ ID NO: 50), YKAAAAPA (SEQ ID NO: 51), EKPKVEAYK (SEQ ID NO: 52), KPKVEAYKA (SEQ ID NO: 53), PKVEAYKAA (SEQ ID NO: 54), KVEAYKAAA (SEQ ID NO: 55), VEAYKAAAA (SEQ ID NO: 56), EAYKAAAAP (SEQ ID NO: 57), AYKAAAAPA (SEQ ID NO: 58), EKPKVEAYKA (SEQ ID NO: 59), KPKVEAYKAA (SEQ ID NO: 60), PKVEAYKAAA (SEQ ID NO: 61), KVEAYKAAAA (SEQ ID NO: 62), VEAYKAAAAP (SEQ ID NO: 63), EAYKAAAAPA (SEQ ID NO: 64), EKPKVEAYKAA (SEQ ID NO: 65), KPKVEAYKAAA (SEQ ID NO: 66), PKVEAYKAAAA (SEQ ID NO: 67), KVEAYKAAAAP (SEQ ID NO: 68), VEAYKAAAAPA (SEQ ID NO: 69), EKPKVEAYKAAA (SEQ ID NO: 70), KPKVEAYKAAAA (SEQ ID NO: 71), PKVEAYKAAAAP (SEQ ID NO: 72), KVEAYKAAAAPA (SEQ ID NO: 73), EKPKVEAYKAAAA (SEQ ID NO: 74), KPKVEAYKAAAAP (SEQ ID NO: 75), PKVEAYKAAAAPA (SEQ ID NO: 76), EKPKVEAYKAAAAP (SEQ ID NO: 77), and KPKVEAYKAAAAPA (SEQ ID NO: 78). Properties of some of these peptides and additional suitable peptides are shown in Stern, J. N. H. et al. 2005 Proc Nail Acad Sci USA 102(5):1620, which is hereby incorporated in its entirety by reference herein.

Certain embodiments of the invention herein provide an anti-DEC205-J5 fusion complex (SEQ ID NO: 81) and an anti-DEC205-PLP139-151 complex (SEQ ID NO: 82).

An embodiment of the invention herein also provides an anti-33D1-J5 fusion complex that targets a different subset of dendritic cells, CD4+ DC (Dudziak, D. et al. 2007 Science 315: 107).

Without being limited by any particular steric and spatial arrangement of peptide to antibody, the peptide in various embodiments is separated from the antibody by a spacer or linker, for example, a series of amino acid residues, for example, glycines, and/or alanines, and/or serines. For example, about five amino acid residues, for example, six or seven residues, would function to provide spatial separation of the peptide from the antibody molecule.

Further, it is envisioned that a fusion of the antibody protein to multiple copies of one or more species of one or more suitable peptides, for example, iterations of two, three or more copies of J5, or iterations of J5 alternating with another peptide, are within the scope of the compositions here.

### DEFINITIONS

Unless the context otherwise requires, as used in this description and in the following claims, the terms below shall have the meanings as set forth:

The term “autoimmune condition” means a disease state caused by an inappropriate immune response that is directed to a self-encoded entity which is known as an autoantigen.

The term “demyelinating condition” includes a disease state in which a portion of the myelin sheath, consisting of plasma membrane wrapped around the elongated portion of the nerve cell, is removed by degradation. A demyelinating condition can arise post-vaccination, post-anti TNF treatment, post-viral infection, and in MS. Symptoms of MS include weakness, spasticity, fatigue, numbness, pain, ataxia, tremor, depression, speech, vision and cognitive disturbances, dizziness, and bladder, bowel and sexual dysfunction. A form of MS is episodic, each episode followed by a period of remission, with symptoms worsening in each episode (remitting-relapsing), culminating in death.

The term “anergy” means unresponsiveness of the immune system of a subject to an antigen. Similarly, a treatment may be immunosuppressive or tolerogenic, with respect to antigen stimulation, and that the treatment is narrowly tailored or generally non-specific.

The term “subject” means a mammal, preferably a human. The term “patient” refers to a human having an autoimmune disease such as a demyelinating condition, such as MS.

The term “derivative” of an amino acid means a non-naturally occurring chemically related form of that amino acid having an additional substituent, for example, an N-carboxyanhydride group, a γ-benzyl group, an ε,N-trifluoroacetyl group, or a halide group attached to an atom of the amino acid.

The term “analog” means a non-naturally occurring non-identical but chemically related form of the reference amino acid. For example, the analog can have a different steric configuration, such as an isomer of an amino acid having a D-configuration rather than an L-configuration, or an organic molecule with the approximate size and shape of the amino acid, or an amino acid with modification to the atoms that are involved in the peptide bond, so as to be protease resistant when polymerized in the context of a peptide or polypeptide.

The phrases “amino acid” and “amino acid sequence” include without limitation all naturally occurring amino acid molecules and additionally one or more components that are amino acid derivatives and/or amino acid analogs having part or the entirety of the residues for any one or more of the 20 naturally occurring amino acids in that sequence. For example, in an amino acid sequence having one or more tyrosine residues, a portion of one or more of those residues can be substituted with homotyrosine. Further, an amino acid sequence having one or more non-peptide or peptidomimetic bonds between two adjacent residues, is included within this definition.

The term “hydrophobic” amino acid means aliphatic amino acids alanine (A or ala), glycine (G or gly), isoleucine (I or ile), leucine (L or leu), methionine (M or Met), proline (P or pro), and valine (V or val), the terms in parentheses being the one letter and three letter standard code abbreviations for each amino acid, and aromatic amino acids tryptophan (W or trp), phenylalanine (F or phe), and tyrosine (Y or tyr). These amino acids confer hydrophobicity as a function of the length of aliphatic and size of aromatic side chains, when found as residues within a protein.

The term “basic” amino acid means amino acids, histidine (H or his), arginine (R or arg) and lysine (K or lys), which confer a positive (his, lys, and arg) charge at physiological values of pH in aqueous solutions on peptides containing these residues.

Immunomodulatory peptides are shown in U.S. Pat. No. 6,930,168 issued Aug. 16, 2005, U.S. Pat. No. 7,456,252 issued Nov. 25, 2008, and U.S. Pat. No. 7,566,762 issued Jul. 28, 2009, each of these patents incorporated herein by reference in its entirety. The immunomodulatory peptides were designed and were identified by comparison to a test compound using one or more assay methods such as ability to bind to MHC class II protein.

The term “immunosuppressive peptides” refers to peptides such as a peptide 15-mers described in Stern, J. N. H. et al. 2005 Proc Natl Acad Sci 102: 1620, which upon targeting immature developmental stages of DC differentiation induce T-cell anergy or Treg cells. By contrast, DCs transformed into mature DCs by activation stimuli represent immunogenic DCs capable of inciting primary T cells responses. Immunosuppressive peptides are broadly immunosuppressive, i.e., they induce unresponsiveness to a number of different autoantigens. The immunosuppressive peptides act by inducing non-specific regulatory T cells such as Foxp3− (Stem, J. N. J. et al. 2008 Proc Natl Acad Sci 105:5172; Yin et al. 2009 J Neuroimmunology 251:43).

The term “tolerogenic peptides” or “encephalitogenic peptides” refers to peptides that are “self-antigens” deriving from myelin proteins, such as proteolipid protein (PLP), myelin basic protein (MBP), or myelin oligodendrocyte protein (MOG). These peptides, when associated to MHC class II molecules, are the target structure for autoreactive CD4⁺ T cells (Falk, K. et al. 2000 J Exp Med 191: 717). Under certain circumstances the tolerogenic peptides may also induce antigen-specific tolerance. These peptides act mainly by anergizing autoreactive T cells and by inducing antigen-specific regulatory T cells such as Foxp3+.

The term “surfaces of MHC class II HLA-DR2 protein” includes the portions of the protein molecule in its 3-dimensional configuration that are in contact with its external environment. For example, the surfaces include amino acid residues found in features of the protein that interact with aqueous solvent and are capable of binding to other cell components such as nucleic acids, other proteins, and peptides.

The term “antigen binding groove” refers to a three dimensional antigen interactive site on the surface of the MHC class II protein molecule (Stern, L. J. et. al. 1994 Nature 368:215) that is formed by surfaces of the α and β subunits of the MHC protein molecule.

The term “immunomodulator” includes a substance (e.g. a drug) which has an effect on the immune system. For example, immunomodulators include immunosuppressants and immunostimulants that decrease or increase immune responses, respectively.

The term “immunosuppressant” includes any substance that results in decreasing an immune response, or suppressing amount of function of the immune system. The substance may be exogenous, as immunosuppressive drugs, or endogenous, as produced testosterone. General broad suppression of the immune system function leads to increased susceptibility to infectious disease and cancer. Immunosuppressants are prescribed under conditions for which normal immune response is undesirable, such as for an autoimmune disease, or after organ or tissue transplant.

The term “immunostimulant” includes any substance, such as a drug or a nutrient that stimulates the immune system by inducing activation or increasing activity of any of its components. For example, female sex hormones are known to stimulate both adaptive and innate immune responses. Certain autoimmune diseases such as lupus erythematosus strike women preferentially, and onset often coincides with puberty.

The term “oligomer” includes a series of a plurality of peptide units, covalently linked, for example, by peptide bonds. The term “homo-oligomer” includes an oligomer in which the sequence unit that is repeated is identical in all units. The term “hetero-oligomer” includes an oligomer in which the peptide units that are repeated are not identical in amino acid sequence.

The term “flexible molecular linker” includes linkers that have backbone lengths of about 50-80 Å, extending, for example to about 540 Å, to about 750 Å, or greater. If composed of amino acids residues, the linker may contain about 10-20 residues, about 20-50 residues, or about 50-125 residues.

Linkers can include components other than amino acids, for example, the linkers can include a polymer or a copolymer of organic acids, aldehydes, alcohols, thiols, and/or amines; polymers or copolymers of hydroxy-, amino, and/or di-carboxylic acids; a polymer or a copolymer of saturated or unsaturated hydrocarbons; a polymer or a copolymer of naturally and non-naturally occurring amino acids. Exemplary linkers are described in PCT/US97/13885 (Feb. 12, 1998), which is hereby incorporated herein by reference. The fusion proteins herein accordingly, are constructed herein to contain at least one copy, or a plurality of copies of the peptide. The amino acid sequences of peptide can be separated by a linker from the amino acid sequence of the antibody protein. FIG. 1 is a drawing of a composition provided herein which is a fusion of the J5 peptide or the PLP139-151 peptide to the carboxy terminus of the H chain. It is within the scope of the compositions of the invention herein to have additional copies of the peptide, or for the amino acid sequences of peptides used in fusions herein, to have added or deleted amino acids. Further, fusions of the peptide may be to the L chain and/or to terminus of the H or L chain.

While the peptides herein are referred to as “synthetic”, for multiple reasons such as cost, the ease of preparation, ability to introduce non-naturally occurring amino acids and non-peptidic bonds, and high state of purity of materials produced by peptide synthesis, it is also possible to synthesize the materials herein by expression of a nucleic acid encoding the peptide, particularly for longer forms such as oligomers and polymers. Such recombinantly produced peptides, oligomers and polymers are readily prepared by one of ordinary skill in the recombinant genetic arts, and are within the embodiments of the present invention. The synthesis of peptides is described in U.S. Pat. No. 6,930,168. Genetic construction is described in U.S. Pat. No. 6,930,168, U.S. Pat. No. 7,456,252 and U.S. Pat. No. 7,566,762 incorporated herein by reference in entirety.

The term “antibody” as referred to herein includes whole antibodies and any antigen binding fragment (i.e., “antigen-binding portion”) or single chains thereof, for example, Fv fragments. A naturally occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antigen portion”), as used herein, refers to full length or one or more fragments of an antibody that retain the ability to specifically bind to a target (e.g., DEC205). It has been shown that the antigen-binding function of an antibody can be performed by fragments of a full-length antibody. Examples of binding fragments encompassed within the term “antigen-binding portion” of an antibody include a Fab fragment, a monovalent fragment consisting of the V_{L}, V_{H}, C_{L} and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al. 1989 Nature 341:544), which consists of a V_{H} domain; and an isolated complementarity determining region (CDR).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird R. E. et al. 1988 Science 242:423; and Huston, J. S. et al. 1988 Proc Natl Acad Sci USA 85:5879). Such single chain antibodies are also intended to be encompassed within the term “antigen-binding portion” of an antibody. These antibody fragments are obtained using conventional techniques known to those of skill in the art, and the fragments are screened for utility in the same manner as are intact antibodies.

An “isolated antibody”, as used herein, refers to an antibody that is substantially free of other antibodies having different antigenic specificities (e.g., an isolated antibody that specifically binds DEC205 is substantially free of antibodies that specifically bind antigens other than DEC205). An isolated antibody that specifically binds DEC205 may, however, have cross-reactivity to other antigens, such as DEC205 molecules from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences. The human antibodies of the invention may include amino acid residues not encoded by human sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM, IgE, IgG such as IgG1 or IgG4) that is provided by the heavy chain constant region genes.

The phrases “an antibody recognizing an antigen” and “an antibody specific for an antigen” are used interchangeably herein with the term “an antibody which binds specifically to an antigen.”

As used herein, an antibody or an antibody-fusion protein that specifically binds to a dendritic cell receptor, e.g., to a target DEC205 which is specifically a human target, is intended to refer to an antibody that binds to the human DEC205 with a K_{D} of about 5×10⁻⁹ M or less, about 2×10⁻⁹ M or less, or about 1×10⁻¹ M or less. An antibody that “cross-reacts with an antigen other than human DEC205” is intended to refer to an antibody that binds that antigen with a K_{D} of about 0.5×10⁻⁸ M or less, about 5×10⁻⁹ M or less, or about 2×10⁻⁹ M or less. An antibody that “does not cross-react with a particular antigen” is intended to refer to an antibody that binds to that antigen, with a K_{D} of about 1.5×10⁻⁸ M or greater, or a K_{D} of about 5-10×10⁻⁸ M or about 1×10⁻⁷ M or greater. In certain embodiments, such antibodies that do not cross-react with the antigen exhibit essentially undetectable binding against these proteins in standard binding assays.

As used herein, an antibody that inhibits binding of a target to the DEC205 receptor refers to an antibody that inhibits a target binding to the receptor with a K of about 1 nM or less, about 0.75 nM or less, about 0.5 nM or less, or about 0.25 nM or less. GL117 is a bacterial anti-β-galactosidase nonspecific isotype-matched rat monoclonal antibody negative control (Hawiger, D. et al. 2001 J Exp Med 194: 769).

The term “K_{assoc}” or “Kₐ”, as used herein, is intended to refer to the association rate of a particular antibody-antigen interaction, whereas the term “K_{dis}” or “K_{D},” as used herein, is intended to refer to the dissociation rate of a particular antibody-antigen interaction. The term “K_{D}”, as used herein, is intended to refer to the dissociation constant, which is obtained from the ratio of K_{d} to Kₐ (i.e. K_{d}/Kₐ) and is expressed as a molar concentration (M). K_{D} values for antibodies can be determined using methods well established in the art. A method for determining the K_{D} of an antibody is by using surface plasmon resonance, or using a biosensor system such as a Biacore® system.

As used herein, the term “affinity” refers to the strength of interaction between antibody and antigen at single antigenic sites. Within each antigenic site, the variable region of the antibody “arm” interacts through weak non-covalent forces with antigen at numerous sites; the more interactions, the stronger the affinity.

As used herein, the term “avidity” refers to an informative measure of the overall stability or strength of the antibody-antigen complex. It is controlled by three major factors: antibody epitope affinity; the valence of both the antigen and antibody; and the structural arrangement of the interacting parts. Ultimately these factors define the specificity of the antibody, that is, the likelihood that the particular antibody is binding to a precise antigen epitope.

As used herein, the term “cross-reactivity” refers to an antibody or population of antibodies binding to epitopes on other antigens. This can be caused either by low avidity or specificity of the antibody or by multiple distinct antigens having identical or very similar epitopes. Cross reactivity is sometimes desirable when one wants general binding to a related group of antigens or when attempting cross-species labeling when the antigen epitope sequence is not highly conserved in evolution.

As used herein, the term “high affinity” for an IgG antibody refers to an antibody having a K_{D} of 10⁻⁸ M or less, 10⁻⁹ M or less, or 10⁻¹⁰ M or less for a target antigen. However, “high affinity” binding can vary for other antibody isotypes. For example, “high affinity” binding for an IgM isotype refers to an antibody having a K_{D} of 10⁻⁷ M or less, or 10⁻⁸ M or less.

Standard assays to evaluate the binding ability of the antibodies toward DEC205 of various species are known in the art, including for example, ELISAs, western blots and RIAs. Suitable assays are described in detail in the Examples. The binding kinetics (e.g., binding affinity) of the antibodies also can be assessed by standard assays known in the art, such as by Biacore analysis. Assays to evaluate the effects of the antibodies on functional properties of DEC205 (e.g., receptor binding, preventing or ameliorating autoimmune disease) are described in further detail in the Examples.

Accordingly, an antibody that “inhibits” one or more of these DEC-205 functional properties (e.g., biochemical, immunochemical, cellular, physiological or other biological activities, or the like) as determined according to methodologies known to the art and described herein, will be understood to relate to a statistically significant decrease in the particular activity relative to that seen in the absence of the antibody (e.g., or when a control antibody of irrelevant specificity is present). An antibody that inhibits a DEC-205 activity effects such a statistically significant decrease by at least 10% of the measured parameter, by at least 50%, 80% or 90%, and in certain embodiments an antibody of the invention may inhibit greater than 95%, 98% or 99% of DEC-205 functional activity.

The term “dendritic cell” includes immature and mature myeloid progenitor cells and cells derived from these that are capable of differentiating into dendritic cells.

The term “DEC205 receptor” (DEC205) refers to DEC205 protein as naturally expressed by cells and variants of DEC205 (e.g., human DEC205, GENBANK Accession number AAC17636, or mouse DEC205, GENBANK Accession number AAK81722). Antibody compositions specific for human DEC205 are known and are commercially available. The amino acid sequence of anti-human DEC205 is found in GENBANK Accession number ABD72617 (SEQ ID NO: 79; Table 1).

TABLE 1
An amino acid sequence of a human anti-DEC205 antibody

(SEQ ID NO: 79)

| | |
|---|---|
| 1 | mgwsciilfl vatatgvhsq vqiefalgkp ipnpllglds tsrsgraand pftivhgntg |
| 61 | kcikpvygwi vaddcdeted klwkwvsqhr lfhlhsqkcl glditksvne lrmfscdssa |
| 121 | mlwwkcehhs lygaaryrla lkdghgtais nasdvwkkgg seeslcdqpy heiytrdgns |
| 181 | ygrpcefpfl idgtwhhdci ldedhsgpwc attlnyeydr kwgiclkpen gcednwekne |
| 241 | qfgscyqfnt qtalswkeay vscqnqgadl lsinsaaelt ylkekegiak ifwiglnqly |
| 301 | sargwewsdh kpinflnwdp drpsaptigg sscarmdaes glwqsfscea qlpyvcrkpl |
| 361 | nntveltdvw tysdtrcdag wlpnngfcyl lvnesnswdk ahakckafss dlisihslad |
| 421 | vevvvtklhn edikeevwig lkniniptlf qwsdgtevtl tywdenepnv pynktpncvs |
| 481 | ylgelgqwkv qsceeklkyv ckrkgeklnd assdkmcppd egwkrhgetc ykiyedevpf |
| 541 | gtncnitits rfeqeylndl mkkydkslrk yfwtglrdvd scgeynwatv ggrrravtfs |
| 601 | nwnflepasp ggcvamstgk svgkwevkdc rsfkalsick kmsgplgpee aspkpddpcp |
| 661 | egwqsfpasl scykvfhaer ivrkrnweea erfcqalgah lssfshvdei keflhfltdq |
| 721 | fsgqhwlwig lnkrspdlqg swqwsdrtpv stiimpnefq qdydirdcaa vkvfhrpwrr |
| 781 | gwhfyddref iylrpfacdt klewvcqipk grtpktpdwy npdragihgp pliiegseyw |
| 841 | fvadlhlnye eavlycasnh sflatitsfv glkaiknkia nisgdgqkww irisewpidd |
| 901 | hftysrypwh rfpvtfgeec lymsaktwli dlgkptdcst klpficekyn vsslekyspd |
| 961 | saakvqcseq wipfqnkcfl kikpvslfts qasdtchsyg gtlpsvlsqi eqdlitsflp |
| 1021 | dmeatlwigl rwtayekink wtdnreltys nfhpllvsgr lripenffee esryhcalil |
| 1081 | nlqkspftgt wnftscserh fvslcqkyse vksrqtlqna setvkylnnl ykiipktltw |
| 1141 | hsakreclks nmqlvsitdp yqqaflsvqa llhnsslwig lfsqddelnf gwsdgkrlhf |
| 1201 | srwaetngql edcvvldtdg fwktvdcndn qpgaicyysg netekevkpv dsvkcpspvl |
| 1261 | ntpwipfqnc cynfiitknr hmattqdevh tkcqklnpks hilsirdeke nnfvleqlly |
| 1321 | fnymaswvml gityrnnslm wfdktplsyt hwragrptik nekflaglst dgfwdiqtfk |
| 1381 | vieeavyfhq hsilackiem vdykeehntt lpqfmpyedg iysviqkkvt wyealnmcsq |
| 1441 | sgghlasvhn qngqlfledi vkrdgfplwv glsshdgses sfewsdgstf dyipwkgqts |
| 1501 | pgncvlldpk gtwkhekcns vkdgaicykp tkskklsrlt yssrcpaake ngsrwiqykg |
| 1561 | hcyksdqalh sfseakklcs khdhsativs ikdedenkfv srlmrennni tmrvwlglsq |
| 1621 | hsvdqswswl dgsevtfvkw enksksgvgr csmliasnet wkkvecehgf grvvckvplg |
| 1681 | pdsssepksc dkthtcppcp apellggpsv flfppkpkdt lmisrtpevt cvvvdvshed |
| 1741 | pevkfnwyvd gvevhnaktk preeqynsty rvvsvltvlh qdwlngkeyk ckvsnkalpa |
| 1801 | piektiskak gqprepqvyt lppsreemtk nqvsltclvk gfypsdiave wesngqpenn |
| 1861 | ykttppvlds dgsfflyskl tvdksrwqqg nvfscsvmhe alhnhytqks 1s1spgksss |
| 1921 | qlsr |

### Autoimmune Diseases

An autoimmune disease results when a host's immune response fails to distinguish foreign antigens from self molecules (autoantigens) thereby eliciting an aberrant immune response. The immune response towards self molecules results in a deviation from the normal state of self-tolerance, which arises when the production of T cells and B cells capable of reacting against autoantigens has been prevented by events that occur in the development of the immune system early in life. The cell surface proteins that play a central role in regulation of immune responses through their ability to bind and present processed peptides to T cells are the major histocompatibility complex (MHC) molecules (Rothbard, J. B. et al. 1991 Annu Rev Immunol 9:527).

Autoimmune diseases include following conditions: autoimmune hemolytic anemia, autoimmune oophoritis, autoimmune thyroiditis, autoimmune uveoretinitis, Crohn's disease, chronic immune thrombocytopenic purpura, colitis, contact sensitivity disease, diabetes mellitus, Graves disease, Guillain-Barre's syndrome, Hashimoto's disease, idiopathic myxedema, multiple sclerosis, myasthenia gravis, psoriasis, pemphigus vulgaris, rheumatoid arthritis, and systemic lupus erythematosus.

Multiple sclerosis (MS) is an inflammatory disease of the central nervous system affecting 0.1% of the population, and is associated in northern European caucasoid MS patients with the HLA-DR-2 (DRB1*1501) haplotype (Olerup, O. et al. 1991 Tissue Antigens 38:1).

An animal model of MS, experimental autoimmune encephalomyelitis (EAE), is a T cell-mediated autoimmune disease. EAE can be induced by subcutaneous injection of peptides derived from myelin components such as myelin basic protein (MBP; Madsen, L. S. et al. 1999 Nat Genet 23:343), proteolipid protein (PLP; Greer, J. M. et al. 1992 R Immunol 149:783) or myelin oligodendrocyte glycoprotein (MOG; Mendel, I. et al. 1995 Eur J Immunol 25:1951). In the course of EAE, autoreactive CD4+ T cells recognize self-antigens presented by murine class II MHC molecules (e.g. H-2As), ultimately leading to pathological changes that can be monitored as clinical signs of disease. EAE provides a well studied system for testing the efficacy of potential therapeutic compounds to suppress the disease. These compounds have included cytokines (Leonard, J. P. et al. 1996 Ann N Y Acad Sci 795: 216), peptide antigens that induce anergy (Gaur, A. et al. 1992 Science 258: 1491) or that induce oral tolerance (Kennedy, K. J. et al. 1997 R Immunol 159:1036; Weiner, H. L. 1997 Immunol Today 18:335), or altered peptide ligands (Pfeiffer, C. et al. 1995 J Exp Med 181:1569; Nicholson, L. B. et al. 1997 Proc Natl Acad Sci USA 94: 9279).

A number of therapeutic agents have been developed to treat autoimmune diseases. For example, agents have been developed that can prevent formation of low molecular weight inflammatory compounds by inhibiting a cyclooxygenase. Also, agents are available that can function by inhibiting a protein mediator of inflammation by sequestering the inflammatory protein tumor necrosis factor (TNF) with an anti-TNF specific monoclonal, antibody fragment, or with a soluble form of the TNF receptor. Finally, agents are available that target and inhibit the function of a protein on the surface of a T cell (the CD4 receptor or the cell adhesion receptor ICAM-1) thereby preventing interaction with an antigen presenting cell (APC). However, compositions which are natural folded proteins as therapeutic agents can incur problems in production, formulation, storage, and delivery. Further, natural proteins can be contaminated with pathogenic agents such as viruses and prions.

An additional target for inhibition of an autoimmune response is the set of lymphocyte surface proteins represented by the MHC molecules. Specifically, these proteins are encoded by the MHC class II genes designated as HLA (human leukocyte antigen)-DR, -DQ and -DP. Each of the MHC genes is found in a large number of alternative or allelic forms within a mammalian population. The genomes of subjects affected with certain autoimmune diseases, for example, MS and rheumatoid arthritis (RA), are more likely to carry one or more characteristic MHC class II alleles, to which that disease is linked.

A potential source of agents for treatment of MS and other demyelinating conditions is to identify peptides that bind selectively in vitro to a purified MI-IC class II allele protein molecule, particularly to a protein which is a product of an MHC class II allele associated with demyelinating conditions. In addition, the agent should bind to that protein as it occurs on the surfaces of antigen presenting cells in vivo, and thereby block, anergize, or inactivate the class of T cells that are responsible for the demyelinating conditions, such as MS.

Major candidates for target antigens in MS include myelin basic protein (MBP), proteolipid protein (PLP), and myelin oligodendrocyte glycoprotein (MOG). T cells reactive with these antigens have been found both in normal blood (Wucherpfennig, K. W. et al. 1994 J Immunol 150:5581; Steinman L. et al. 1995 Mol Med Today 1:79) and in MS patients (Wucherpfennig, K. W. et al. 1991 Immunol Today 12:227; Marcovic-Plese, S. et al. 1995 J Immunol 155:982; Correale, J. et al. 1995 Neurology 45:1370; Kerlero de Rosbo, N. et al. 1997 Eur J Immunol 27:3059; Tsuchida, T. et al. 1994 Proc Natl Acad Sci USA 91:10859), suggesting that autoreactive T cells may be involved in the pathogenesis of the disease, such that these cells once activated can penetrate the blood-brain barrier. Microbial agents have been suggested to provide potential stimuli for induction of MS by immunological cross-reaction with MBP (Wucherpfennig, K. W. et al. 1995 Cell 80:695; Brocke, S. et al. 1993 Nature 365:642).

Studies indicate that MBP is an important target antigen in the immunopathogenesis of MS. MBP-specific T cells have been shown to be clonally expanded in MS patients and in an in vivo activated state (Wucherpfennig, K. W. et al. 1994 J Immunol 150:5581; Allegretta, M. et al., 1990 Science 247:718; Ota, K. et al. 1990 Nature 346:183; Zhang, J. et al. 1994 J Exp Med 179:973). Reactivity with the immunodominant MBP 84-102 peptide is found predominantly in subjects carrying HLA-DR2, a genetic marker for susceptibility to MS. Structural characterization of MBP 84-102 identified residues critical for MHC class II binding and for TCR recognition (Wucherpfennig, K. W. et al. 1994 J Exp Med 179:279), which have been recently confirmed by the crystal structure of HLA-DR2 complexed with MBP 85-99 peptide (Smith, K. J. et al. 1998 J Exp Med 19:1511).

Demyelinating conditions have been found to occur post-viral infection, post-vaccination, post-encephalomyelitis (Wucherpfennig, K. W. et al. 1991 Immunol Today 12:277) and following administration of certain anti-TNF agents (FDA Talk Paper, Food and Drug Administration Public Health Service, Rockville, Md.).

The activity of Cop1 appears to involve, as a first step, binding to the surface of antigen-presenting cells (APC), for example to class II MHC proteins (Fridkis-Hareli, M. et al. 1994 Pro Natl Aca Sci USA 91:4872), following which its effectiveness may be due either to competition with myelin antigens (for example, MBP, PLP, MOG) for activation of specific effector T cells recognizing peptide epitopes derived from these proteins (Ben-Nun, A. et al. 1996 J Neurol 243:S14-22; Teitelbaum, D. et al. 1996 J Neuroimmunol 64:209), and/or induction of antigen-specific regulatory T cells (Aharoni R. et al. 1993 Eur J Immunol 23:17).

Examination of additional therapeutic agents and investigation of the mechanisms involved in their activities could potentially result in information that could lead to improved therapeutic reagents. Recent studies have shown that virtually all of the large variety of copolymers found in the random mixture of YEAK bound to purified molecules of each of human HLA-DR1, -DR-2 and -DR4 molecules, showing that YEAK generally binds to purified class II MHC proteins (Fridkis-Hareli, M. et al. 1998 J Immunol 160:4386). Cop1 further competes for binding of MBP 85-99 to HLA-DR-2 (DRB1*1501) and inhibits responses of DR-2-restricted T cells to MBP 85-99. Study of the binding to class II MHC molecules of random copolymers containing only 3 of the 4 amino acids of Cop1, for example, YAK, revealed that YAK is the most effective (Fridkis-Hareli, M. et al. 1999 Int Immunol 11:635).

The binding motif of Cop1 to the MS-associated molecule HLA DR-2 (DRB1*1501) shows E at P-2, K at P-1 and Y at P1, with no preferences observed at other positions (Fridkis-Hareli, M. et al. 1999 J Immunol 162:4697). Further, A is overrepresented at P1. As P1 is the anchor position, binding of Y at this position was not anticipated. The P1 pocket in proteins encoded by the DR-2 allele is small (due to the presence of β86Val rather than β86Gly), and overrepresentation of A at this position may result from this fact. The effect of K at P-1 appears to be due to stabilization of binding by the interaction of K with residues in the top of the al helix, similarly to residue K at P-1 of HA 306-318 (SEQ ID NO: 5) complexed with HLA-DR1 which can interact with the side chains of α1 helix residues at Sα53 or Eα55 (Stern, L. J. et al. 1994 Nature 368:215).

### Therapeutic Compositions

A pharmaceutically acceptable carrier includes any and all solvents, dispersion media, coatings, antimicrobials such as antibacterial and antifungal agents, isotonic and absorption delaying agents and the like that are physiologically compatible. Preferably, the carrier is suitable for intravenous (i.v.), intramuscular (i.m.), oral, intraperitoneal (i.p.), transdermal, or subcutaneous (s.c.) administration, and the active compound can be coated in a material to protect it from inactivation by the action of acids or other adverse natural conditions.

The methods of the invention include incorporation of a fusion protein as provided herein into a pharmaceutical composition suitable for administration to a subject. A composition of the present invention can be administered by a variety of methods known in the art as will be appreciated by the skilled artisan. The active compound can be prepared with carriers that will protect it against rapid release, such as a controlled release formulation, including implants, transdermal patches, and microencapsulated delivery systems. Many methods for the preparation of such formulations are patented and are generally known to those skilled in the art. See, e.g., Sustained and Controlled Release Drug Delivery Systems, J. R. Robinson, Ed., Marcel Dekker, Inc., NY, 1978. Therapeutic compositions for delivery in a pharmaceutically acceptable carrier are sterile, and are preferably stable under the conditions of manufacture and storage. The composition can be formulated as a solution, microemulsion, liposome, or other ordered structure suitable to high drug concentration.

Dosage regimens can be adjusted to provide the optimum desired response (e.g., a therapeutic response). For example, a single bolus or oral dose can be administered, several divided doses can be administered over time, or the dose can be proportionally reduced or increased as indicated by the exigencies of the disease situation.

In general, an embodiment of the invention is to administer a suitable daily dose of a therapeutic fusion protein composition that will be the lowest effective dose to produce a therapeutic effect, for example, mitigation of symptoms. The therapeutic fusion protein compounds of the invention are preferably administered at a dose per subject per day of at least about 2 mg, at least about 5 mg, at least about 10 mg or at least about 20 mg as appropriate minimal starting dosages. In general, the compound of the effective dose of the composition of the invention can be administered in the range of about 50 to about 400 micrograms of the compound per kilogram of the subject per day.

A physician or veterinarian having ordinary skill in the art can readily determine and prescribe the effective dose of the pharmaceutical composition required. For example, the physician or veterinarian could start doses of the compound of the invention employed in the pharmaceutical composition at a level lower than that required in order to achieve the desired therapeutic effect, and increase the dosage with time until the desired effect is achieved.

In another embodiment, the pharmaceutical composition includes also an additional therapeutic agent. Thus in a method of the invention the pharmaceutical fusion protein composition can be administered as part of a combination therapy, i.e. in combination with an additional agent or agents. Examples of materials that can be used as combination therapeutics with the fusion protein for treatment of autoimmune disease as additional therapeutic agents include: an antibody or an antibody fragment that can bind specifically to an inflammatory molecule or an unwanted cytokine such as interleukin-6, interleukin-8, granulocyte macrophage colony stimulating factor, and tumor necrosis factor-α; a monoclonal antibody that can bind specifically to the cellular adhesion molecule α4-integrin such as natalizumab; an enzyme inhibitor which can be a protein, such as α₁-antitrypsin, or aprotinin; an enzyme inhibitor which can be a cyclooxygenase inhibitor; an enzyme inhibitor which can be a type II topoisomerase inhibitor such as mitoxantrone; an engineered binding protein, for example, an engineered protein that is a protease inhibitor such an engineered inhibitor of a kallikrein; an antibacterial agent, which can be an antibiotic such as amoxicillin, rifampicin, erythromycin; an antiviral agent, which can be a low molecular weight chemical, such as acyclovir; a steroid, for example a corticosteroid, or a sex steroid such as progesterone; a non-steroidal anti-inflammatory agent such as aspirin, ibuprofen, or acetaminophen; an anti-cancer agent such as methotrexate, cis-platin, 5-fluorouracil, or adriamycin; a cytokine blocking agent; an adhesion molecule blocking agent; or a cytokine; or an immunosuppressive drug such as fingolimod (FTY720, Novartis Pharma), sphingosine-1-phosphate receptor modulator, as shown in U.S. Pat. No. 6,476,004, which is incorporated herein by reference in its entirety.

An additional therapeutic agent can be a cytokine, which as used herein includes without limitation agents which are naturally occurring proteins or variants and which function as growth factors, lymphokines, interferons particularly interferon-β, tumor necrosis factors, angiogenic or antiangiogenic factors, erythropoietins, thrombopoietins, interleukins, maturation factors, chemotactic proteins, or the like. An additional agent to be added to a fusion protein composition that is an embodiment of the invention herein can be a copolymer, for example, Copaxone® which is a YEAK or Cop 1, or a copolymer comprising a subset of these or other amino acids (Aharoni, R. et al. WO 00/05250, PCT/US99/16747), or an oligopeptide or peptide derivative (Strominger, J. et al. WO 00/05249, PCT/US99/16617; WO 02/59143, PCT/US02/02071), or a copolymer FYAK comprising amino acids tyrosine, phenylalanine, alanine and lysine (Fridkis-Hareli, M. et al. 2002 J Clin Invest 109:1635; U.S. Pat. No. 7,381,790 issued Jun. 3, 2008). Additional therapeutic agents to be used in combination with a composition of the invention and which are cytokines include interferon-β, interleukin-4 and interleukin-10.

A therapeutic agent to be used with the composition of the invention can be an engineered binding protein, known to one of skill in the art of remodeling a protein that is covalently attached to a virion coat protein by virtue of genetic fusion (Ladner, R. et al., U.S. Pat. No. 5,233,409; Ladner, R. et al., U.S. Pat. No. 5,403,484), and can be made according to methods known in the art. A protein that binds any of a variety of other targets can be engineered and used in the present invention as a therapeutic agent in combination with a heteropolymer of the invention.

An improvement in the symptoms as a result of such administration is noted by a decrease in frequency of recurrences of episodes of the autoimmune condition such as MS, by decrease in severity of symptoms, and by elimination of recurrent episodes for a period of time after the start of administration. A therapeutically effective dosage preferably reduces symptoms and frequency of recurrences by at least about 20%, for example, by at least about 40%, by at least about 60%, and by at least about 80%, or by about 100% elimination of one or more symptoms, or elimination of recurrences of the autoimmune disease, relative to untreated subjects. The period of time can be at least about one month, at least about six months, or at least about one year.

Methods of use of fusion proteins having sequences provided herein can be the basis of treating other autoimmune diseases which are associated with HLA-DR gene products, by competing with candidate autoantigens for binding to these protein receptor molecules, or by inducing T cell anergy or even T cell apoptosis, or by suppression of T cells, such that subsequent T cell response to an autoantigen is inhibited in vivo. Further, fusion proteins having within the sequence one or more additional components, such as amino acid analogs or derivatives added in varying quantities into the polymerization reaction, can be effective inhibitors of a variety of autoimmune T cell responses.

Those skilled in the art will recognize or be able to ascertain using routine experimentation the numerous equivalents to the examples and claims herein, which are exemplary and are not intended to be further limiting. The contents of all references cited throughout the application are hereby incorporated by reference.

### EXAMPLES

The following Materials and Methods were used throughout the examples.

### Example 1

### Mice

SJL/J (H-2^{s}) mice were purchased from the Jackson Laboratory (Bar Harbor, Me.). Six to 12 week old, female mice were used in all experiments. Animals were maintained at the animal facilities of Harvard University according to the animal protocol guidelines of Harvard Medical School, Boston, Mass. Vβ6⁺ PLP139-151-specific 5B6 TCR transgenic mice on the rag^{-/-}B10.S (B10/I-A⁵) background along with nontransgenic rag^{-/-}B10.S mice were previously described and were used in PLP examples (Waldner, H. et al. 2004 J Clin Invest 113:990). Humanized mice previously described (Stern, J. N. H. et al. 2004 Proc Natl Aca Sci USA 101:11743) are similar to another double-transgenic mouse (Madsen, L. S. et al. 1999 Nat Genet 23:343) and may also be used.

### Example 2

### Protein and Peptide Synthesis

Peptides and proteins were synthesized as described previously (Fridkis-Hareli, M. et al. 2001 Hum Immunol 62:753).

### Example 3

### Generation of Fusion Antibodies Targeting the DEC205 Receptor

Examples herein analyzed a nucleotide sequence encoding a fusion of J5 peptide or PLP139-151 peptide to the heavy chain of anti-DEC205 C-terminus (FIG. 1). The peptides herein are exemplary only and not further limiting. The fused peptide was observed to be a tolerogenic peptide or an immunosuppressiv peptide rather than an antigenic peptide and, without being limited by any particular theory or mechanism of action, induces tolerance by a mechanism that is different from that of produced by antigenic peptides.

To prepare DEC205 and J5-specific antibody, double-stranded DNA fragments were constructed using synthetic oligonucleotides. DNA fragments were added in-frame to the C terminus of the heavy chains of cloned NLDC-145 (DEC205 specific).

To prepare DEC205 and PLP139-151-specific antibody, double-stranded DNA fragments coding for PLP139-151 with spacer residues on both sides were constructed using synthetic oligonucleotides, according to Kretschmer, K. et al. 2006 Nat Protoc 1:653. The following oligonucleotides were used: PLP-1 forward, 5′-cta geg aca tgg cca aga agg aga cag tct gga ggc tcg agg agt tcg gta ggt tea caa aca ggC AT; PLP-1 reverse, 5′CAG GC Tat gcc tgt ttg tga acc tac cga act cct cga gcc tee aga ctg tct cct tct tgg cca tgt cg; PLP-2 forward, 5′-AGC CTG GGC AAA TGG CTG GGC CAT CCG GAT AAA TTT tat tat gac ggt agg aca tga tag gc; PLP-2 reverse, 5′-ggc cgc eta tea tgt cct acc gtc ata ata AAA TTT ATC CGG ATG GCC CAG CCA TTT GCC (the PLP139-151 peptide-encoding nucleotide sequence split between the two sets of oligonucleotides is shown in uppercase letters). DNA fragments were added in-frame to the C terminus of the heavy chains of cloned NLDC-145 (DEC205 specific) and III/10 isotype control constant regions.

To ensure the specificity of antigen targeting the rat IgG2a, constant regions of the original NLDC-145 and isotype control antibodies were replaced with mouse IgG1 constant regions, which carry point mutations interfering with Fc receptor binding (Clynes, R. A. et al. 2000 Nat Med 6:443). The plasmid vectors of the IgH chain cDNA of the cloned NLDC-145 (pDEC-IgH) and GL117 (GL117/10-IgH) and their respective IgL-k light chain cDNA (pDEC-IgL-k and pGL117/10-IgL-k) were used herein. The plasmid vectors containing the cDNA of amino acids 107-119 of HA (HA107-119) added to the C terminus of cloned αDEC205 and III/10 control were produced according to Kretschmer, K. et al. 2005 Nat Immunol 6:1219.

Hybrid antibodies were produced using the FreeStyle MAX 293 expression system (Invitrogen, CA) according to the manufacturer's recommendations. In brief, suspension cultures of FreeStyle 293-F cells were maintained in serum-free FreeStyle 293 expression medium and transiently transfected with plasmid vectors of the respective IgH chain and Igk chain cDNA using FreeStyle MAX reagent. The original DEC205 specific antibody NLDC-145 (without peptide tag), which was included in some experiments as a control, was produced by hybridoma cells in serum-free Hybridoma medium (Invitrogen, CA). All antibodies were purified on prepacked HiTrap™ Protein G HP columns (Amersham Biosciences, Piscataway, N.J.). Protein concentrations were determined spectophotometrically by measuring the absorbance at 280 nm. The amount and the presence of full-length recombinant fusion protein were verified by SDS/PAGE with an IgG1/IgLκ antibody as a reference.

### Example 4

### Generation and Analysis of PLP139-151-Specific T-Cell Line

SJL/J mice were immunized with 75 μg of PLP 139-151 emulsified in CFA (Difco Laboratories), and T-cell lines were established as previously described (Stem, J. N. et al. 2008 Proc Natl Acad Sci USA 105:5172). T-cell lines were stained with monoclonal antibodies and aVβ screening kit using FACSCalibur with CellQuest software (all from BD Biosciences, Franklin Lakes, N.J.). Cell sorting was performed using MoFlo (Daco, Hamburg, Germany).

### Example 5

### Proliferation Assay

CD11c⁺ dendritic cells (DCs) were isolated from SJL splenocytes with magnetic beads using a CD11c⁺ selection kit from Miltenyi Bioscience (Bergisch Gladbach, Germany). Purified DCs were washed with PBS twice and incubated with 1 μg fusion antibodies αDEC205/-PLP and GL117-PLP for three hours in complete DMEM media in 37° C. After incubation, DCs were washed twice with complete DMEM media and plated at 5×10³ cells per well together with 1×10⁵ PLP 139-151-specific T-cell lines in 96-well plates. After four days of co-culture, plates were pulsed with 1 μCi ³H thymidine per well for 18 hours. Proliferation was detected as described previously (Stern, J. N. et al. 2008 Proc Natl Acad Sci USA 105:5172). The Vβ6-positive PLP139-151-specific T cells were isolated from PLP139-151-specific T-cell transgenic mice splenocytes on an SJL background. SJL DCs were prepared as described above and incubated with 1,000, 300, 100, or 30 ng of fusion antibodies (DEC205-PLP specific monoclonal antibodies, DEC-HA specific monoclonal antibodies, and GL117-PLP specific monoclonal antibodies). DCs were washed twice with complete DMEM and plated at 5×10³ cells per well together with 1×10⁵ Vβ6⁺ PLP139-151-specific TCR tg T cells in 96-well plates. Proliferation was detected as described previously (Stern, J. N. et al. 2008 Proc Natl Acad Sci USA 105:5172).

### Example 6

### Cytokine Measurement Using Cytokine Bead Arrays and Luminex

Splenocytes from SJL mice preimmunized with fusion antibodies (DEC205-PLP-specific monoclonal antibodies, DEC205-HA specific monoclonal antibodies, DEC205 specific monoclonal antibodies, and Gl117-PLP specific monoclonal antibodies) were restimulated with PLP139-151 at different concentrations. Cytokines were detected in the supernatants, obtained from the proliferation assays described above by either Cytometric Bead Array (CBA) kit (BD Biosciences Pharmingen, Franklin Lakes, N.J.) according to the instruction manual or the Luminex core facility in Baylor College of Medicine. The samples were analyzed by BD FACSCalibur using BD CellQuest, BD CBA Software, and Luminex software.

### Example 7

### IL-17 ELISPOT Assay

SJL/J mice were preimmunized with 1 μg i.p. of DEC205-PLP specific monoclonal antibodies, DEC205-HA specific monoclonal antibodies, or GL117-PLP specific monoclonal antibodies ten days before inducing EAE. Mice were then immunized s.c. with 75 μg of PLP139-151 emulsified in CFA, and 200 ng of pertussin toxin (PT) was administered i.v. the next day. Splenocytes were prepared 17 d after disease induction, and 2×10⁵ cellswere plated per well on pre-coated IL-17 ELISPOT plates (eBiosciences, San-Diego, Calif.). Splenocytes were stimulated overnight with 10 μg/mL PLP139-151. Unstimulated wells were used as controls. ELISPOT plates were processed and developed according to the manufacturer's protocol. Well images were acquired, and spots were analyzed using an automated ELISPOT counter. Spots per million were calculated by multiplying the average of triplicate wells (2×10⁵) by 5-fold.

### Example 8

### Adoptive Transfer of CD4⁺ T Cells from Anti-DEC205-Fusion Immunized SJL Mice

SJL/J mice were preimmunized (day 10, 15, or 20) with 1 μg i.p. of fusion monoclonal antibodies (DEC205-PLP specific monoclonal antibodies, DEC205-HA specific monoclonal antibodies, DEC205 specific monoclonal antibodies alone, or GL117-PLP specific monoclonal antibodies). SJL mice were then immunized s.c. with 75 μg of PLP139-151 peptide, and the next day, 200 ng of PT (List Biological Laboratories, Campbell, Calif.) was given i.v. MACS beads (Miltenyi Biotec, Bergisch Gladbach, Germany) were used to purify CD4⁺ T cells from SJL mice. CD4⁺ T-cell purity ranged from 87% to 93% after CD4-negative selection enrichment. CD4⁺ T cells (5×10⁶) were injected i.v. into naïve six to eight week-old SJL/J mice along with 75 μg (s.c.) of PLP139-151 and 200 ng (i.v.) of PT the next day. The mice were scored daily for 30 days.

### Example 9

### Adoptive Transfer of Vβ6⁺ 5B6 TCR Transgenic CD4⁺ T Cells from B10.S Mice

MACS beads (Miltenyi Biotec, Bergisch Gladbach, Germany) were used to purify CD4⁺ T cells from Vβ6⁺ 5B6 TCR transgenic B10.S mice (Waldner, H. et al. 2004 J Clin Invest 113:990). CD4⁺ T-cell purity ranged from 84% to 90% after CD4-negative selection enrichment. The 10×10⁶ T cells were injected i.v. into naïve eight week-old B10.S rag^{-/-} mice along with 1 μg (i.p.) of fusion antibodies (DEC205-PLP specific monoclonal antibodies or GL117-PLP specific monoclonal antibodies). Splenocytes were removed ten days later. Single cell suspensions were stimulated with PLP139-151 for four days. Proliferation assay was performed as described (Stern, J. N. et al. 2008 Proc Natl Acad Sci USA 105:5172). The Vβ6⁺ TCR trangsenic CD4⁺ T cells were stimulated by cross-linking using plate bound CD3/CD28 (BD Biosciences, Franklin Lakes, N.J.) antibodies coated overnight to detect cytokine production. Three days after stimulation, supernatants were removed, and cytokines were measured by Luminex assay as described above. FACS analysis was carried out using CD4-FITC and Foxp3-PE (BD Biosciences, Franklin Lakes, N.J.).

### Example 10

### Effect of Fusion Antibodies on the Induction of EAE

To determine the therapeutic effect of peptide-antibody fusions on appearance and progression of the mouse model disease EAE in subjects used herein, eight to 12 week old female SJL mice were pre-immunized subcutaneously (s.c.) before inducing EAE, each with nothing (control), or 1 μg of DEC205-J5-specific monoclonal antibodies, or 1 μg of GL117-J5 (control fusion antibody made from a nonspecific isotype-matched rat monoclonal antibody control), or 300 μg of J5 (Stern, J. N. et al. 2005 Proc Natl Acad Sci USA 102(5):1620). Ten days later each of mice was administered s.c. 75 μg of PLP139-151 emulsified in CFA, followed on the next day with 200 ng i.v. of pertussis toxin (List Biological Laboratories, Campbell, Calif.). The mice were monitored for appearance of clinical signs of EAE daily, and were scored from 0-5 as follows: 1, limp tail; 2, hind limb paralysis; 3, complete hind limp paralysis; 4, four limbs paralyzed; 5, moribund. All scoring was performed double blind.

For preimmunization, SJL/J mice were immunized with 1 μg i.p. of fusion antibodies (DEC205-PLP specific monoclonal antibodies, DEC205-HA specific monoclonal antibodies, DEC205-specific monoclonal antibodies alone, or GL117-PLP-specific monoclonal antibodies) either ten or fifteen days before inducing EAE. Six- to ten week-old female mice were immunized s.c. with 75 μg of PLP139-151 emulsified in CFA; 200 ng of pertussis toxin (PT; List Biological Laboratories, Campbell, Calif.) was given i.v. on the day after immunization. The mice were monitored for clinical signs of EAE, and they were scored from 0 to 5:1, limp tail; 2, hind limb paralysis; 3, complete hind limb paralysis; 4, four limbs paralyzed; 5, moribund. All scoring was performed double blind.

### Example 11

### Anti-DEC205-J5 Fusion Protein Ameliorates EAE Induced by PLP 139-151 in SJL/J Mice

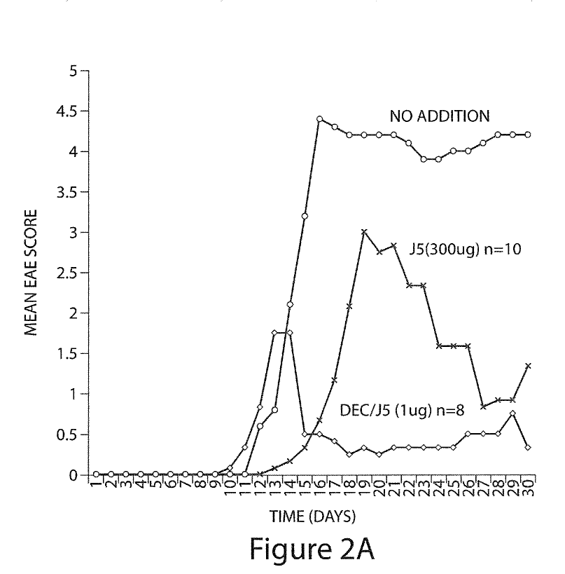

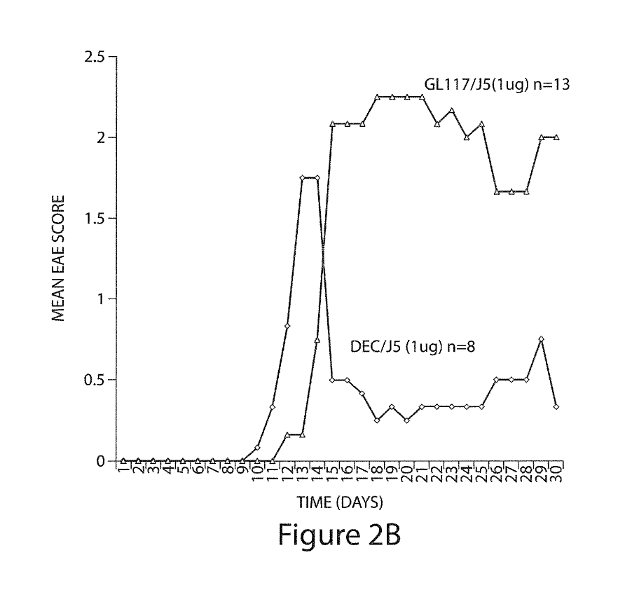

The first sign of EAE appeared at day 10 and reached a maximum mean score of 4.5 by day 16 (FIG. 2 panel A). The severity of EAE was monitored in each group of five different groups of six mice each, as shown in FIG. 2 panels A and B. The groups were administered the following treatments prior to induction of EAE: anti-DEC205-J5 fusion (1 μg; closed diamonds, shown in both panels A and B), control GL117-J5 fusion (1 μg; closed triangles, panel B), J5 (300 μg; asterisks, panel A) together with PLP139-151, or control (no treatment; closed circles, panel A). Each group was treated at day 10 with PLP139-151 (75 μl in CFA) followed the next day with 200 ng i.v. of pertussis toxin to induce EAE.

The data show that the severity of EAE disease symptoms in subjects pre-immunized with J5 (FIG. 2 panel A; asterisks) was only moderately suppressed, having peak symptoms on a scale of 3, compared to 4.5 for control mice receiving no therapeutic agents. Subjects receiving GL117-J5 fusion (FIG. 2 panel B; closed triangles) displayed substantial disease symptoms with a score greater than 2.

In contrast, subjects immunized with anti-DEC205-J5 fusion developed only a mild disease (clinical score less than 1; FIG. 2 panels A and B; closed diamonds). No mortalities were observed for subjects pre-immunized with anti-DEC205-J5 fusion (FIG. 2 panel A; closed diamonds). Further, the data show that treatment with anti-DEC205-J5 fusion was the most efficient treatment to reduce or eliminate the symptoms of EAE, as merely 1 μg of this material was more effective treatment for reducing symptoms than 300 μg of J5 peptide per se.

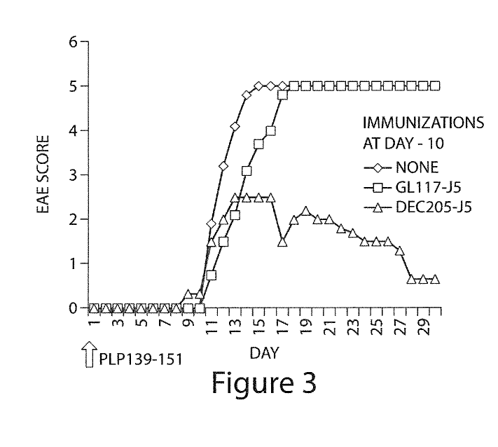

An additional data obtained using anti-DEC205-J5 fusion protein to specifically ameliorate PLP139-151-induced EAE in SJL mice (FIG. 3) corrected a potential anomaly shown in FIG. 2 panel B). The isotype control antibody GL117-J5 appeared to ameliorate disease to some extent, average scores 2.0-2.5, as compared to average scores of 4.0-4.5 in untreated control, and score of 0.5 in mice treated with anti-DEC205-J5. New data herein indicated that little or no amelioration was induced by the GL117-J5 control. The possible anomaly in FIG. 2 panel B may have been due to the failure of PLP139-151 to induce disease in several mice of the GL117-J5 control group (score 0.0), thus lowering the average score. Such anomalies sometimes result from failure to inject pertussis toxin i.v. into the tail vein accurately. Most important, theconclusions from both data sets show that DEC-205-J5 fusion significantly reduced severity of EAE symptoms to a level comparable to elimination of symptoms.

The amount of DEC205-J5 fusion protein administered to reduce symptoms, 1 μg (FIGS. 2 and 3) contains a proportional J5 content of approximately 20 nanograms. The fusion protein delivered the J5 portion of the fusion protein directly to the dendritic cell receptors. Without being limited by any particular theory or mechanism of action, delivery of J5 as a portion of the fusion herein produced a more effective therapeutic regimen because the fusion composition precluded hydrolysis of isolated peptide in serum in vivo by aminopeptidase digestion. The time course herein yielded data indicating that tolerance induced in this way lasted at least two to three weeks. Therefore, it may be possible that only two injections per month would protect a multiple sclerosis patient from additional relapses of the disease. With present therapeutic regimes, MS patients require a daily injection of a relatively ineffective amino acid copolymer (Copaxone) to reduce the frequency of relapses.

### Example 12

### Dendritic Cell Targeting of Proteolipid Protein-Derived Peptide Using DEC205 Specific Fusion Antibodies

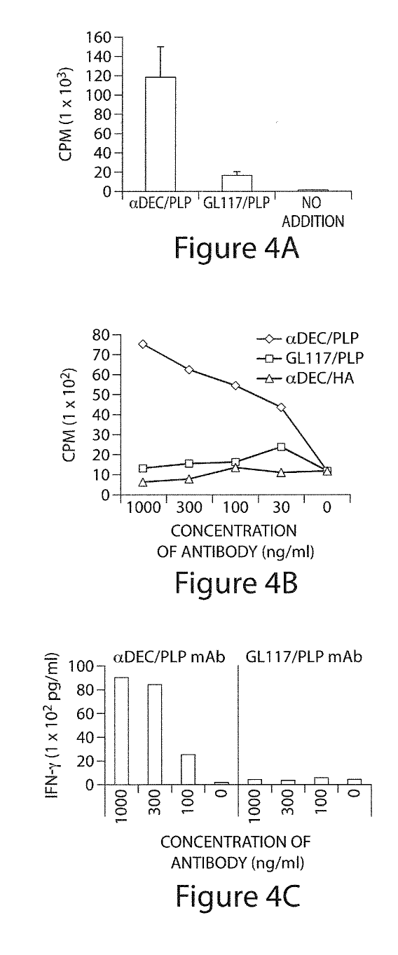

To target the encephalogenic antigen to DCs, recombinant proteins consisting of amino acids 139-151 of proteolipid protein (PLP139-151) fused either to the C terminus of the Ig heavy chain of cloned anti-DEC205 (αDEC205-PLP) or to the GL117 isotype control antibody (GL117-PLP) were produced. To confirm that the antigenic peptide delivered by the anti-DEC205 fusion antibody was properly processed and presented, purified splenic CD11c⁺ DCs from SJL mice were incubated for three hourswith various concentrations of either αDEC205-PLP or GL117-PLP control antibodies. After unbound antibodies were removed by extensive washing, DCs were cocultured with antigen-specific CD4⁺ Vβ6⁺ T cells from PLP139-151-specific Vβ6⁺ TCR transgenic mice (Waldner, H et al. 2004 J Clin Invest 113:990; Kuchroo, V. K. et al. 2002 Annu Rev Immunol 20:101). ³H-thymidine incorporation at day 4 of the culture demonstrated that DCs preincubated with αDEC205-PLP fusion antibody induced vigorous proliferation of these transgenic T cells compared with GL117-PLP isotype control antibody or in the absence of a specific antigen (FIG. 4 panel A).

In addition, a PLP139-151-specific T-cell line was established by immunizing SJL mice with PLP139-151 and restimulating splenocytes from the immunized mice with the same peptide three times at two-week intervals in vitro. The CD4⁺ T-cell line obtained exhibited an activated surface marker phenotype (CD25⁺, CD69⁺, CD45⁺, CD30⁺, GITR⁺, CTLA4⁺, CD71^{low}, or CD62L^{low}) and secreted high amounts of IL-17 (10,300 pg/mL) along with IL-6 (1,300 pg/mL), IL-5 (772 pg/mL), GM-CSF (2,960 pg/mL), and TNF-α (278 pg/mL). Coculture of the PLP139-151 T-cell line with CD11c⁺ DCs preincubated with 1 μg of anti-DEC205/-PLP fusion antibody substantially enhanced T-cell proliferation in a dose-dependent manner (FIG. 4 panel B). In contrast, preincubation of DCs with either GL117-PLP isotype control antibody or anti-DEC205 antibody fused to an irrelevant antigen (peptide 107-119 of hemagglutinin, HA; αDEC205/HA) induced little proliferation. Proliferation was accompanied by an about 10-fold increase in IFN-γ secretion only after treatment with anti-DEC205-PLP (FIG. 4 panel C).

### Example 13

### Immunization or Preimmunization with DEC205-PLP Specific Antibodies Ameliorates EAE Induced by Either Adoptive Transfer of a PLP139-151-Apecific T-Cell Line or by Immunization with PLP139-151

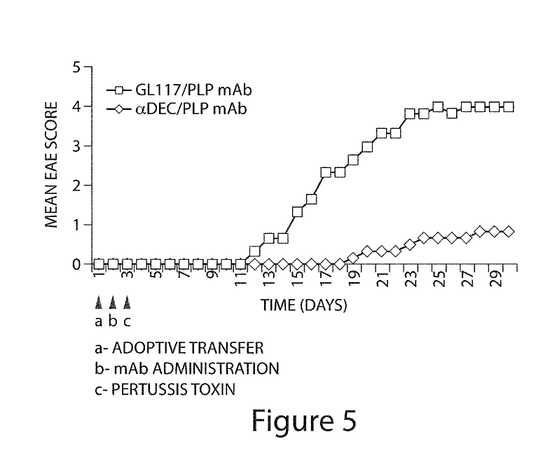

The PLP139-151-specific splenic T-cell line shown in Examples herein was adoptively transferred into naïve SJL mice to passively induce EAE, and then the mice were injected with either 1 μg of DEC205-PLP-specific or control GL117/PLP-specific fusion antibodies, equivalent to about 20 ng of PLP139-151. All recipients were injected with pertussis toxin (PT) the following day. As expected, mice that received PLP139-151-specific T cells and the GL117-PLP isotype control antibody rapidly developed severe EAE with a maximal mean score of 4 on day 28 of this experiment (FIG. 5). In contrast, mice that received PLP139-151 specific T cells followed by immunization with anti-DEC205-PLP exhibited a substantially delayed onset of disease with a low maximal mean score of 1 on day 28. Thus, anti-DEC205-mediated targeting of nanogram amounts of PLP139-151 efficiently interfered with the passive induction of EAE by adoptive transfer of highly encephalitogenic T cells with the same antigen specificity.

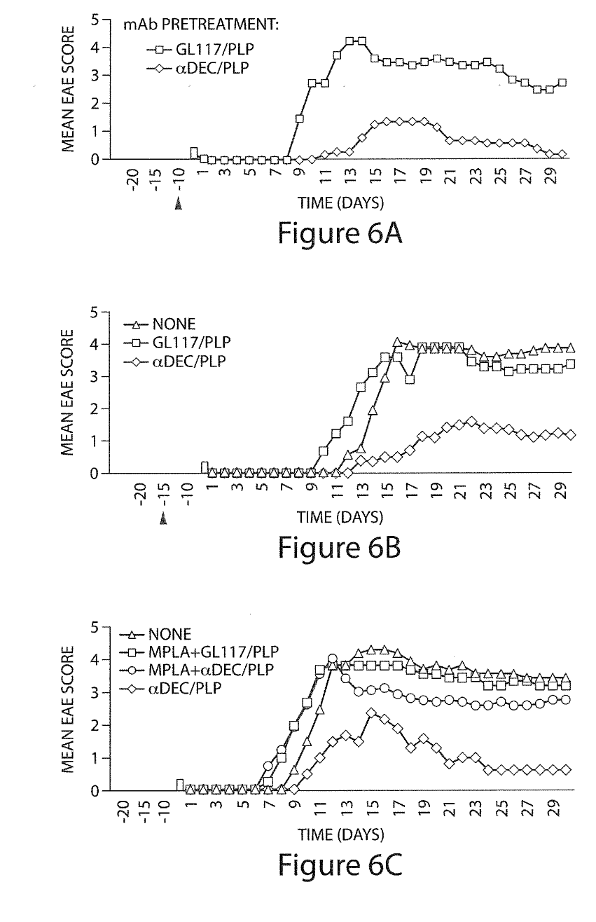

To determine whether preimmunization of SJL mice with DEC205-PLP-specific antibodies also ameliorated disease induced in mice immunized with unconjugated PLP139-151, SJL mice were either left untreated or treated with a single injection of 1 μg of DEC205-PLP or GL117-PLP isotype control antibody at day minus 10 or minus 15. EAE was induced on day 0 by injection of 75 μg PLP139-151 in CFA followed by 200 ng PT (PLP139-151/CFA/PT) the next day, and mice were monitored daily for 30 d for clinical signs of EAE (FIG. 6 panels A, B and C). When EAE was induced in naïve SJL mice (i.e., without pretreatment), all of the mice developed clinical symptoms between days 9 and 10 and rapidly progressed to severe EAE with mean maximum scores of 3.8-4.4 by days 16-18 (FIG. 6 panels A and B). Similarly, pretreatment with the GL117-PLP control resulted in severe EAE with scores of 3.6-4.4 on days 16-18. Deaths of 40-60% of the mice occurred in these experiments. Thus, pretreatment with GL117-PLP control antibody did not result in an amelioration of disease progression and severity and was comparable to non-pretreated mice. Other control antibodies, anti-DEC205 itself (NLDC-145), and recombinant anti-DEC205-HA107-119 also had no significant effect on the disease course.

By contrast, mice pretreated with 1 μg anti-DEC205-PLP showed consistently delayed onset of disease by up to 5 days, with maximal scores of 1.4-1.7 on days 16-23 (FIG. 6 panels A and C; only two mortalities were observed). This reduction was seen when anti-DEC205/PLP was administered 10 or 15 days before induction of EAE (FIG. 6 panels A and B) but in one experiment appeared less effective when administered at day 20. Thus, the treatment prevented disease when administered 23 days before disease onset in controls. However, administration of anti-DEC205-PLP at the same time as immunization with PLP139-151/CFA/PT did not interfere with onset or severity of EAE, possibly due to the rapid conversion of immature to mature DCs by immunization. Moreover, coadministration at day 10 of 1 μg anti-DEC205-PLP with 10 μg monophosphoryl lipid A (MPLA), a low-toxicity derivative of LPS with potent proinflammatory activity that leads to DC maturation and activation (Mata-Haro, V. et al. 2007 Science 316:1628), completely abrogated the beneficial effect of αDEC205/PLP alone on PLP139-151/CFA/PTinduced EAE (FIG. 6 panel C).

### Example 14

### Effect of Anti-DEC205-Mediated Targeting on Pathogenic IL-17-Producing T Cells

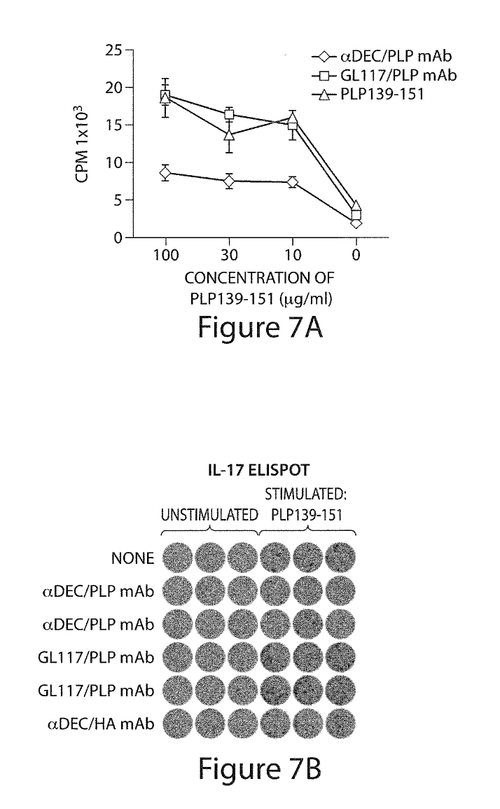

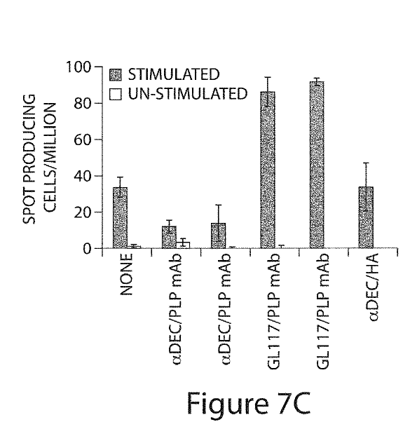

To determine whether anti-DEC205-PLP-mediated targeting interfered with early antigen-specific T-cell induction, SJL mice were either left untreated or treated with a single injection of 1 μg of anti-DEC205-PLP or GL117-PLP control monoclonal antibodies ten days before immunization with PLP139-151/CFA/PT. Total splenocytes that contained both antigen-presenting cells and T cells isolated from mice at day 17, either without pretreatment or pretreated with GL117-PLP control antibody, proliferated vigorously to various PLP139-151 concentrations in vitro, whereas little proliferation was seen after pretreatment with anti-DEC205-PLP even in response to nonphysiologically high peptide concentrations (FIG. 7 panel A). Thus, anti-DEC205 targeting in vivo reduced either the numbers of antigen-specific T cells or their proliferative capacity tested in vitro.

To address this question in more detail, the number of pathogenic IL-17-secreting cells in splenocytes from SJL mice that were either left untreated or pretreated with a single injection of 1 μg of recombinant DEC205-PLP specific, GL117-PLP specific control, or irrelevant DEC205-HA specific fusion monoclonal antibodies followed by PLP139-151/CFA/PT immunization ten days later, was determined. ELISPOT analysis at day 17 using total splenocytes and overnight restimulation with varying concentrations of PLP139-151 in vitro showed that anti-DEC205-PLP resulted in an about 2- to 3-fold reduction in the number of cells secreting IL-17 compared with mice that were not pretreated (P<0.02) or were pretreated with anti-DEC205-HA (P<0.03) (FIG. 7 panels B and C). Pretreatment with GL117-PLP seemed to increase the number of IL-17 secreting cells in the spleen (P<0.004).

### Example 15

### CD4⁺ T Cells from Anti-DEC205-PLP-Pretreated Mice Control EAE Induction After Adoptive Transfer

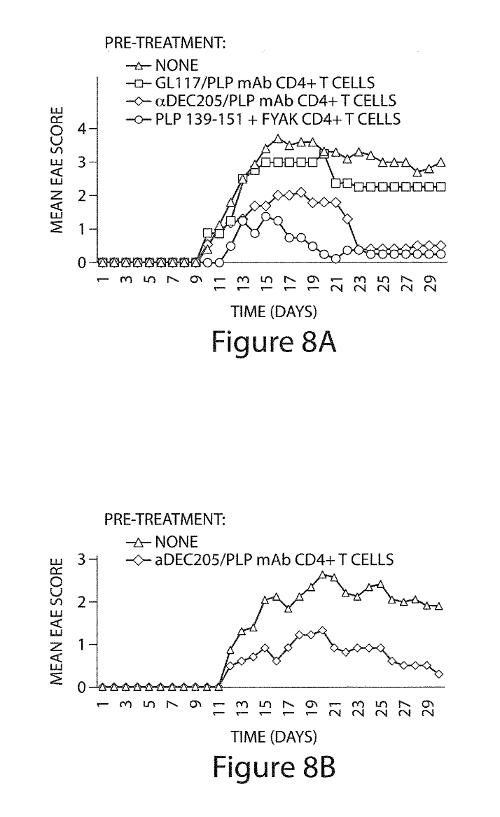

To address the question whether anti-DEC205-PLP-mediated targeting also result in induction of regulatory T cells such as Treg, SJL mice were either untreated or pretreated with either 1 μg anti-DEC205-PLP or GL117-PLP (FIG. 8 panel A). In one of the experiments, as a positive control, an additional group of SJL mice was coimmunized with 500 μg of the synthetic amino acid copolymer poly(F,Y,A,K)n, to ameliorate PLP139-151-induced EAE by the generation of IL-10-secreting Trl-like Tregs (Stern, J. N. et al. 2008 Proc Natl Acad Sci USA 105:5172; Stern, J. N. et al. 2004 Proc Natl Acad Sci USA 101:11743). Disease was induced ten days later by PLP139-151/CFA/PT administration. After an additional ten days, splenic CD4⁺ T cells from all four groups were purified using magnetic beads, and 5×10⁶ cells were i.v. transferred into naïve SJL mice. EAE was induced in recipients the following day by PLP139-151/CFA/PT immunization. Recipients adoptively transferred with 5×10⁶ CD4⁺ T cells from mice without pretreatment or pretreated with GL117-PLP developed severe EAE with mean maximum scores of 3.2-3.6 on days 16-18 (FIG. 8). Adoptive transfer of CD4⁺ T cells from poly (F,Y,A,K)n pretreated mice efficiently prevented EAE induction in recipient SJL mice. Similarly, CD4⁺ T cells from anti-DEC205-PLP-treated mice also significantly ameliorated EAE with a mean maximum score of 2.0 on days 16-18 (P=0.003 compared with the control groups). Surprisingly, symptoms ameliorated in the treated groups (but not in the untreated groups) so that, from day 23 onward, basically no signs of EAE were detectable (FIG. 8). Thus, the generation of regulatory CD4⁺ T cells also played a role in amelioration of EAE after administration of anti-DEC205-PLP.

### Example 16

### Effects of Anti-DEC205-PLP on Pathogenic Vβ6⁺ TCR Transgenic T Cell

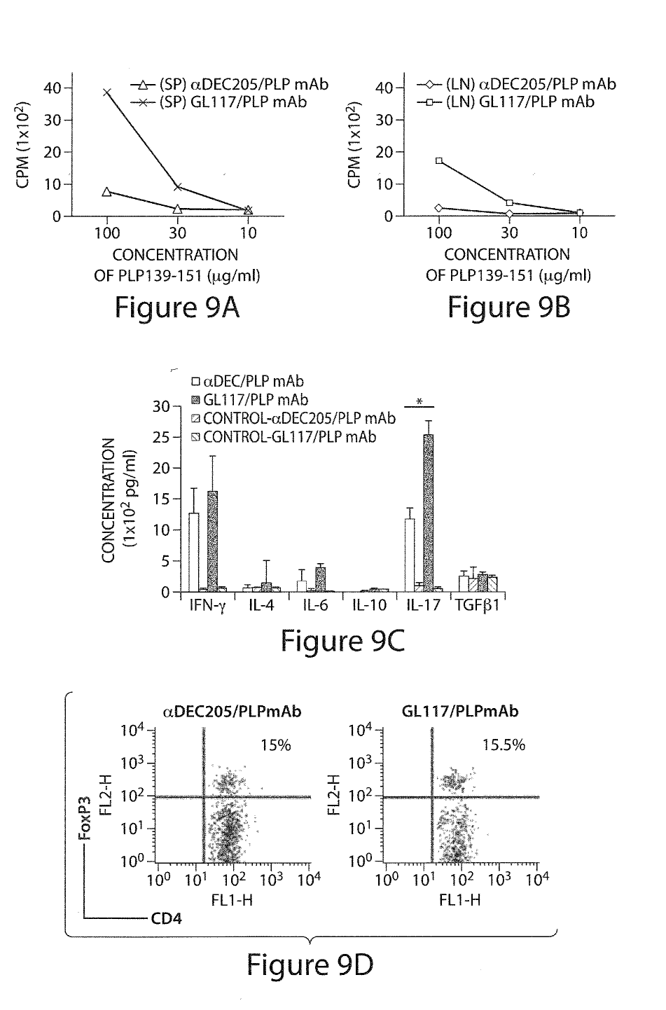

Splenocytes and lymph node cells from Vβ6⁺ TCR CD4⁺ T cells recognizing PLP139-151 obtained from 5B6 transgenic B10.S mice (Waldner, H. et al. 2004 J Clin Invest 113:990; Kuchroo, V. K. et al. 2002 Annu Rev Immunol 20:101) were adoptively transferred into rag^{-/31} B10.S(I-A⁵) mice. Mice were treated with 1 μg of either anti-DEC205-PLP or GL117-PLP. Splenocytes and lymph nodes were harvested ten days later, and CD4⁺ T cells were separated using anti-CD4 magnetic beads. Cells from the mice that had been injected with anti-DEC205-PLP exhibited limited proliferation and reduced IL-17 production but unchanged IFN-γ production in response to in vitro restimulation, in comparison with PLP139-151-specific CD4⁺ T cells from GL117-PLP-treated recipients (P<0.006; FIG. 9 panels A, B and C). Thus, anti-DEC205-PLP targeting in vivo contributed to amelioration of EAE by reducing the number of antigen-specific pathogenic IL-17-producing T cells and their proliferative capacity in vitro. In addition, Foxp3⁺ cells in the CD4⁺ T cell populations were enumerated by FACS (FIG. 9 panel D). The percentage of Foxp3⁺ cells among CD4⁺ cells in anti-DEC205-PLP and GL117-PLP pretreated mice was 15% in each case under these conditions. Anti-DEC205-PLP did not result in detectable conversion of CD4⁺ Foxp3⁻ T cells to Foxp3⁺ cells. The percentage of these cells in normal B10.S mice that have been shown to express a high level of CD4⁺ CD25⁺ Tregs (Reddy, J. et al. 2004 Proc Natl Acad Sci USA 101:15434) averaged 6.1%, and in B10.S mice bearing the Vβ6 TCR transgene, it averaged 8.3%. Thus, homeostatic expansion of Foxp3⁺ CD4⁺ T cells (Jameson, S. C. 2002 Nat Rev Immunol 2: 547; Nishio, J. et al. 2010 J Exp Med, in press) in the rag^{-/-} background likely accounts for the increased numbers found in both anti-DEC205-PLP and anti-GL117-PLP-treated mice. A smaller specific conversion to Foxp3⁺ CD4⁺ T cells induced by anti-DEC205-PLP treatment (Kretschmer, K. et al. 2005 Nat Immunol 6:1219) would not have been detected. CD5 was found to be expressed in a previous study of anti-DEC205-MOG35-55 treatment in an EAE model in C57BL/6 mice (Hawiger, D et al. 2004 Immunity 20:695). However, no CD5 was expressed on the isolated anergized Vβ6⁺ CD4⁺ T cells from anti-DEC205-PLP-treated mice shown here.

### Example 17

### EAE Induction was Prevented in Anti-DEC205-PLP-Treated Subjects

Lack or loss of tolerance to several self-molecules that have been identified as target antigens in autoimmune diseases is one of the key events promoting autoimmunity such as multiple sclerosis or type I diabetes. Despite many studies in both rodents and humans to stimulate tolerogenic mechanisms using various protocols of antigen administration with antigens in different pharmaceutical forms (e.g., peptides or whole antigens) and testing diverse administration routes, robust data demonstrating clinical benefits are not yet available (Kretschmer, K. et al. 2005 Nat Immunol 6:1219). Recent studies in mice have also indicated that repeated administration of free antigens can induce fatal autoimmune responses (Pugliese, A. et al. 2001 J Clin Invest 107: 555). The ability to target minute amounts of antigens to steady-state immature DCs in vivo is an important approach to obtain antigen-specific immunological tolerance.

In earlier studies of immunological tolerance induced by targeting of peptides to immature DCs by fusion to anti-DEC205, several different mechanisms have been reported. In earlier studies using an artificial system in which HA was the target antigen, the induction of immunological unresponsiveness by deletion of autoreactive T cells or by anergization was emphasized (Hawiger, D. et al. 2001 J Exp Med 194:769; Hawiger, D et al. 2004 Immunity 20:695; Bruder, D. et al. 2005 Diabetes 54:3395). Later studies, however, focused on the generation of regulatory T cells as an important mechanism in induction of antigen-specific tolerance (Kretschmer, K. et al. 2005 Nat Immunol 6:1219, Yamazaki, S. et al. 2008 J Immunol 181:6923). In the only previous study using a known autoantigen, MOG35-55-induced EAE in C57BL/6 mice was ameliorated by pretreatment at day minus 7 with anti-DEC205-MOG35-55 (Hawiger, D. et al. 2004 Immunity 20:695). In the present experiment, DEC205-PLP139-151 specific fusion monoclonal antibodies were synthesized and used to prevent EAE in the model in which disease is induced by PLP139-151 in SJL mice. Anti-DEC205-mediated targeting of low nanogram amounts of the immunodominant PLP139-151 efficiently ameliorated EAE induced either by immunization with PLP139-151 or by adoptive transfer of PLP139-151-specific T cells (FIG. 6). It is important to note that, in the PLP139-151-induced EAE model in SJL mice, pretreatment with large doses of free peptide in the absence of adjuvants does not lead to protection from disease induced by subsequent challenge with peptide/CFA/PT, in contrast to the MOG35-55-induced EAE model in C57BL/6 mice (Hawiger, D. et al. 2004 Immunity 20:695). Thus, the fact that anti-DEC205 targeting is several magnitudes more efficient in inducing T-cell responses compared with free peptide administration does not explain the tolerogenic effect of small amounts of anti-DEC205-PLP fusion antibodies in the PLP-induced EAE model.

Anti-DEC205-mediated targeting herein interfered with early antigen-specific T-cell induction in peripheral lymphoid organs upon active EAE induction, reflected by reduced numbers of pathogenic antigen-specific IL-17-producing T cells (FIG. 7). In addition, the remaining cells exhibited an anergic phenotype upon restimulation in vitro. Both deletion and induction of an anergic phenotype in pathogenic T cells contributed to anti-DEC205-PLP-mediated amelioration of EAE.

In addition, however, adoptively transferred CD4⁺ T cells from anti-DEC205-PLP-treated mice efficiently prevented EAE induction in recipients (FIG. 8 panels A and B). These data point toward an additional dominant T-cell suppressive mechanism of immunological tolerance promoted by anti-DEC205-PLP-mediated targeting. However, this experiment does not make clear to what extent de novo generation or expansion of preexisting Foxp3⁻ expressing CD4⁺ Tregs or IL-10 secreting T cells, or conversion of pathogenic C4⁺ Foxp3⁻ cells mediated by anti-DEC205-PLP, contributes to disease amelioration.

To approach the latter possibility, pathogenic CD4⁺ Vβ6⁺ T cells were adoptively transferred to B10.S rag^{-/-} mice. After treatment with anti-DEC205-PLP, splenocytes or lymph node cells were markedly anergic to PLP139-151 and had severely reduced IL-17 production but little or no change in IFNγ secretion. This example reinforces the relative importance of IL-17 in the pathogenesis of EAE in this model system (Axtell, R. C., et al. 2010 Nat Med 16:406-412). A high level of Foxp3⁺ CD4⁺ Vβ6⁺ T cells was seen after treatment with control GL117 specific monoclonal antibodies, and no further increase was found after treatment with anti-DEC205-PLP. Thus, no evidence of specific conversion could be detected under the conditions of the present experiment.

Examples herein demonstrate that anti-DEC205-PLP139-151 ameliorates EAE induction mainly by inducing anergy in PLP139-151-specific T cells. In addition, evidence of T-cell suppression was obtained, although induction of neither IL-10 secretion nor Foxp3⁺ T cells was seen. Hawiger, D. et al., 2004 Immunity 20:695-705, showed MOG35-55 induced EAE was ameliorated by αDEC205/MOG35-55. Additionally, MBP85-99 also induces EAE, and is important in multiple sclerosis (Zhang, J. et al. 1994 J Exp Med 179:3973-3984; Bettelli, E. et al. 2006 J Clin Invest 116:2393-2402). Combination of these three anti-DEC205 fusion proteins represents a therapeutic modality for this disease.

### Example 18

### Fusion Compositions Provide Superior Treatment of MS

Examples herein showed that fusion complexes of the monoclonal antibody DEC205 and/or 33D1 located on distinct sets of dendritic cells with the immunosuppressive peptide J5 or PLP139-151 provide a powerful way to induce tolerance to induction of EAE in mice. Exceedingly small amounts of the fusion complexes suffice to induce tolerance and terminate relapses in the relapsing, remitting model of EAE (Table 2).

TABLE 2
Comparison for treatment of MS of known copolymers, peptide
15mers, and fusion compositions provided herein

| | | mouse | | | | total human | | |
|---|---|---|---|---|---|---|---|---|
| compound | frequency | dose | | human dose | | dose/2 weeks | | side effects |
| Copaxone ® | daily | 150 | μg | 20 | mg | 280 | mg | severe injection |
| | | | | | | | | site pain leading to |
| | | | | | | | | intolerance |
| FYAK | weekly | 50 | μg | (10 | mg)^{a} | (20 | mg)^{a} | none known or |
| (PI-2301) | | | | (3 | mg)^{a} | (6 | mg)^{a} | predicted |

| | | | | | | |
|---|---|---|---|---|---|---|
| peptide 15mer | n.d.^{b} | 300 | μg^{c} | n.d. | n.d.^{b} | none known or |
| | | | | | | predicted |

| | | | | | |
|---|---|---|---|---|---|
| αDEC205-J5 | biweekly | 1 μg = ~20 | (0.2 mg-4 mg = ~4 | (0.2 mg-4 mg = ~4 | none known or |
| fusion protein | | ng J5 | μg-80 μg J5)^{d} | μg-80 μg J5)^{d} | predicted |

^{a}estimated based on Phase 1b clinical trial
^{b}not determined
^{c}See Example 11, and FIG. 2 panel B
^{d}extrapolated from the mouse data

These data would provide the basis for developing a new therapy for the treatment of relapsing, remitting MS and to protect patients from subsequent relapses of the disease. The proposed therapy aims to increase the pool of regulatory T cells in mice and, later, in MS patients.

FYAK is being developed for clinical use by Peptimmune, Inc., which licensed the patent from Harvard. It is effective when administered weekly s.c. at the low doses of 3 or 10 mg. In contrast, Copaxon is administered daily s.c. at a dose of 20 mg., i.e., 140 mg/week. Importantly, the anti-DEC205-J5 fusion protein is an even more potent drug with the same effects but requiring even smaller amounts and at a lower frequency. Administration weekly or possibly at even lower frequency is important because the pain associated with daily administration of Copaxone results in discontinuance of this therapy by some MS patients and limits the frequency with which it is prescribed.

What is claimed is:

1. A composition comprising a fusion protein having a first amino acid sequence of a monoclonal antibody specific for binding a dendritic cell receptor protein and a second amino acid sequence of an immunosuppressive peptide or a tolerogenic peptide.

2. The composition according to claim 1, wherein the amino acid sequence of the immunosuppressive peptide is selected from the group of:
EKPKVEAYKAAAAPA (SEQ ID NO: 1), EKPK (SEQ ID NO: 2), KPKV (SEQ ID NO: 3), PKVE (SEQ ID NO: 4), KVEA (SEQ ID NO: 5), VEAY (SEQ ID NO: 6), EAYK (SEQ ID NO: 7), AYKA (SEQ ID NO: 8), YKAA (SEQ ID NO: 9), KAAA (SEQ ID NO: 10), AAAA (SEQ ID NO: 11), AAAP (SEQ ID NO: 12), AAPA (SEQ ID NO: 13), EKPKV (SEQ ID NO: 14), KPKVE (SEQ ID NO: 15), PKVEA (SEQ ID NO: 16), KVEAY (SEQ ID NO: 17), VEAYK (SEQ ID NO: 18), EAYKA (SEQ ID NO: 19), AYKAA (SEQ ID NO: 20), YKAAA (SEQ ID NO: 21), KAAAA (SEQ ID NO: 22), AAAAP (SEQ ID NO: 23), AAAPA (SEQ ID NO: 24), EKPKVE (SEQ ID NO: 25), KPKVEA (SEQ ID NO: 26), PKVEAY (SEQ ID NO: 27), KVEAYK (SEQ ID NO: 28), VEAYKA (SEQ ID NO: 29), EAYKAA (SEQ ID NO: 30), AYKAAA (SEQ ID NO: 31), YKAAAA (SEQ ID NO: 32), KAAAAP (SEQ ID NO: 33), AAAAPA (SEQ ID NO: 34), EKPKVEA (SEQ ID NO: 35), KPKVEAY (SEQ ID NO: 36), PKVEAYK (SEQ ID NO: 37), KVEAYKA (SEQ ID NO: 38), VEAYKAA (SEQ ID NO: 39), EAYKAAA (SEQ ID NO: 40), AYKAAAA (SEQ ID NO: 41), YKAAAAP (SEQ ID NO: 42), KAAAAPA (SEQ ID NO: 43), EKPKVEAY (SEQ ID NO: 44), KPKVEAYK (SEQ ID NO: 45), PKVEAYKA (SEQ ID NO: 46), KVEAYKAA (SEQ ID NO: 47), VEAYKAAA (SEQ ID NO: 48), EAYKAAAA (SEQ ID NO: 49), AYKAAAAP (SEQ ID NO: 50), YKAAAAPA (SEQ ID NO: 51), EKPKVEAYK (SEQ ID NO: 52), KPKVEAYKA (SEQ ID NO: 53), PKVEAYKAA (SEQ ID NO: 54), KVEAYKAAA (SEQ ID NO: 55), VEAYKAAAA (SEQ ID NO: 56), EAYKAAAAP (SEQ ID NO: 57), AYKAAAAPA (SEQ ID NO: 58), EKPKVEAYKA (SEQ ID NO: 59), KPKVEAYKAA (SEQ ID NO: 60), PKVEAYKAAA (SEQ ID NO: 61), KVEAYKAAAA (SEQ ID NO: 62), VEAYKAAAAP (SEQ ID NO: 63), EAYKAAAAPA (SEQ ID NO: 64), EKPKVEAYKAA (SEQ ID NO: 65), KPKVEAYKAAA (SEQ ID NO: 66), PKVEAYKAAAA (SEQ ID NO: 67), KVEAYKAAAAP (SEQ ID NO: 68), VEAYKAAAAPA (SEQ ID NO: 69), EKPKVEAYKAAA (SEQ ID NO: 70), KPKVEAYKAAAA (SEQ ID NO: 71), PKVEAYKAAAAP (SEQ ID NO: 72), KVEAYKAAAAPA (SEQ ID NO: 73), EKPKVEAYKAAAA (SEQ ID NO: 74), KPKVEAYKAAAAP (SEQ ID NO: 75), PKVEAYKAAAAPA (SEQ ID NO: 76), EKPKVEAYKAAAAP (SEQ ID NO: 77), and KPKVEAYKAAAAPA (SEQ ID NO: 78); and,
the tolerogenic peptide is selected from the group of: an encephalitogenic peptide derived from at least one protein selected from the group of: a proteolipid protein (PLP), a myelin basic protein (MBP), and a myelin oligodendrocyte protein (MOG); and a peptide comprising amino acid sequence HSLGKWLGHPNKF (SEQ ID NO: 80).

3. The composition according to claim 1, wherein the peptide has a length comprising at least four amino acid residues.

4. The composition according to claim 1, further comprising at least one of: a pharmaceutically acceptable salt; a pharmaceutically acceptable carrier; a pharmaceutically acceptable buffer; and an additional therapeutic agent selected from the group consisting of a cytotoxic agent, an immunosuppressive agent, and a chemotherapeutic agent.

5. The composition according to claim 1, wherein the fusion protein is characterized by a function that is immunomodulatory, wherein the immunomodulatory function comprises inhibition of MHC class II interaction with T cells.

6. The composition according to claim 1, wherein the fusion protein is characterized by a function that is immunosuppressive.

7. The composition according to claim 1, wherein the dendritic cell receptor protein is derived from at least one selected from the group of: a mannose receptor, a toll-like receptor, a DEC205, a CLEC9A, and a 33D1.

8. The composition according to claim 1 present in a unit dose effective for treatment of a subject for an autoimmune condition, wherein the autoimmune condition is selected from the group of: a demyelinating condition such as multiple sclerosis (MS); cell mediated disease; an antibody mediated disease; a condition mediated by a T cell or a natural killer (NK) cell; autoimmune hemolytic anemia; autoimmune oophoritis; autoimmune thyroiditis; autoimmune uveoretinitis; Crohn's disease; chronic immune thrombocytopenic purpura; colitis, contact sensitivity disease; diabetes mellitus; Graves' disease; Guillain-Barre's syndrome; Hashimoto's disease; idiopathic myxedema; myasthenia gravis; psoriasis; pemphigus vulgaris; rheumatoid arthritis; and systemic lupus erythematosus.

9. A kit for treating a subject having an autoimmune disease comprising a fusion protein having a first amino acid sequence of a monoclonal antibody specific for binding a dendritic cell receptor protein and a second amino acid sequence of an immunosuppressive peptide or a tolerogenic peptide, as shown in claim 1, in a pharmaceutically acceptable buffer, a container and instructions for use.

10. A method for treating a subject for an autoimmune disease, comprising:
administering to the subject a fusion protein having a first amino acid sequence from a monoclonal antibody that specifically binds a dendritic cell receptor protein and a second amino acid sequence from an immunosuppressive peptide or a tolerogenic peptide; and,
measuring a decrease in severity or frequency of recurrences of the autoimmune disease or an elimination of at least one symptom of the autoimmune disease.

11. The method according to claim 10, wherein the second amino acid sequence is selected from the group of:
EKPKVEAYKAAAAPA (SEQ ID NO: 1), EKPK (SEQ ID NO: 2), KPKV (SEQ ID NO: 3), PKVE (SEQ ID NO: 4), KVEA (SEQ ID NO: 5), VEAY (SEQ ID NO: 6), EAYK (SEQ ID NO: 7), AYKA (SEQ ID NO: 8), YKAA (SEQ ID NO: 9), KAAA (SEQ ID NO: 10), AAAA (SEQ ID NO: 11), AAAP (SEQ ID NO: 12), AAPA (SEQ ID NO: 13), EKPKV (SEQ ID NO: 14), KPKVE (SEQ ID NO: 15), PKVEA (SEQ ID NO: 16), KVEAY (SEQ ID NO: 17), VEAYK (SEQ ID NO: 18), EAYKA (SEQ ID NO: 19), AYKAA (SEQ ID NO: 20), YKAAA (SEQ ID NO: 21), KAAAA (SEQ ID NO: 22), AAAAP (SEQ ID NO: 23), AAAPA (SEQ ID NO: 24), EKPKVE (SEQ ID NO: 25), KPKVEA (SEQ ID NO: 26), PKVEAY (SEQ ID NO: 27), KVEAYK (SEQ ID NO: 28), VEAYKA (SEQ ID NO: 29), EAYKAA (SEQ ID NO: 30), AYKAAA (SEQ ID NO: 31), YKAAAA (SEQ ID NO: 32), KAAAAP (SEQ ID NO: 33), AAAAPA (SEQ ID NO: 34), EKPKVEA (SEQ ID NO: 35), KPKVEAY (SEQ ID NO: 36), PKVEAYK (SEQ ID NO: 37), KVEAYKA (SEQ ID NO: 38), VEAYKAA (SEQ ID NO: 39), EAYKAAA (SEQ ID NO: 40), AYKAAAA (SEQ ID NO: 41), YKAAAAP (SEQ ID NO: 42), KAAAAPA (SEQ ID NO: 43), EKPKVEAY (SEQ ID NO: 44), KPKVEAYK (SEQ ID NO: 45), PKVEAYKA (SEQ ID NO: 46), KVEAYKAA (SEQ ID NO: 47), VEAYKAAA (SEQ ID NO: 48), EAYKAAAA (SEQ ID NO: 49), AYKAAAAP (SEQ ID NO: 50), YKAAAAPA (SEQ ID NO: 51), EKPKVEAYK (SEQ ID NO: 52), KPKVEAYKA (SEQ ID NO: 53), PKVEAYKAA (SEQ ID NO: 54), KVEAYKAAA (SEQ ID NO: 55), VEAYKAAAA (SEQ ID NO: 56), EAYKAAAAP (SEQ ID NO: 57), AYKAAAAPA (SEQ ID NO: 58), EKPKVEAYKA (SEQ ID NO: 59), KPKVEAYKAA (SEQ ID NO: 60), PKVEAYKAAA (SEQ ID NO: 61), KVEAYKAAAA (SEQ ID NO: 62), VEAYKAAAAP (SEQ ID NO: 63), EAYKAAAAPA (SEQ ID NO: 64), EKPKVEAYKAA (SEQ ID NO: 65), KPKVEAYKAAA (SEQ ID NO: 66), PKVEAYKAAAA (SEQ ID NO: 67), KVEAYKAAAAP (SEQ ID NO: 68), VEAYKAAAAPA (SEQ ID NO: 69), EKPKVEAYKAAA (SEQ ID NO: 70), KPKVEAYKAAAA (SEQ ID NO: 71), PKVEAYKAAAAP (SEQ ID NO: 72), KVEAYKAAAAPA (SEQ ID NO: 73), EKPKVEAYKAAAA (SEQ ID NO: 74), KPKVEAYKAAAAP (SEQ ID NO: 75), PKVEAYKAAAAPA (SEQ ID NO: 76), EKPKVEAYKAAAAP (SEQ ID NO: 77), and KPKVEAYKAAAAPA (SEQ ID NO: 78); and,
the tolerogenic peptide is selected from the group of: an encephalitogenic peptide derived from at least one protein selected from the group of: a proteolipid protein (PLP), a myelin basic protein (MBP), and a myelin oligodendrocyte protein (MOG); and a peptide comprising the amino acid sequence HSLGKWLGHPNKF (SEQ ID NO: 80).

12. The method according to claim 10, wherein the dendritic cell receptor protein is selected from at least one of the group: DEC205, CLEC9A and 33D1.

13. The method according to claim 10, wherein measuring a decrease or elimination further comprises monitoring promoting T-cell anergy and generating suppressor T cells, thereby inducing tolerance.

14. The method according to claim 10, wherein prior to administering, the method further comprises chemically linking the monoclonal antibody and the peptide.

15. The method according to claim 10, wherein prior to administering, the method further comprises engineering a recombinant nucleic acid sequence encoding the first amino acid sequence from a chain of the monoclonal antibody or a fragment thereof and the second amino acid sequence from the peptide; and expressing the recombinant nucleic acid sequence in cells.

16. The method according to claim 15, wherein the recombinant nucleic acid sequence encodes the second amino acid sequence of the peptide as the fusion to the first amino acid sequence of a heavy chain of the antibody C-terminus.

17. The method according to claim 10, wherein prior to administering, the method further comprises producing the monoclonal antibody by a hybridoma cell line.

18. The method according to claim 10, wherein measuring a decrease or elimination further comprises monitoring a symptom of the autoimmune disease selected from: autoimmune hemolytic anemia; autoimmune oophoritis; autoimmune thyroiditis; autoimmune uveoretinitis; Crohn's disease; chronic immune thrombocytopenic purpura; colitis; contact sensitivity disease; diabetes mellitus; Graves' disease; Guillain-Barre's syndrome; Hashimoto's disease; idiopathic myxedema; demyelinating condition for example multiple sclerosis (MS); myasthenia gravis; psoriasis; pemphigus vulgaris; rheumatoid arthritis; and systemic lupus erythematosus.

19. The method according to claim 10, wherein the subject is a mammal.

20. The method according to claim 19, wherein the mammal is a rodent with experimental allergic encephalomyelitis.

21. The method according to claim 20, wherein the rodent is a humanized mouse.

22. The method according to claim 19, wherein the mammal is a human.

23. The method according to claim 22, wherein the human is a patient with MS.

24. The method according to claim 10, wherein administering the fusion protein further comprises administering by a route selected from the group of: intravenous, subcutaneous, intramuscular, and intraperitoneal.

25. The method according claim 10, wherein measuring further comprises, analyzing at least one physiological parameter of the demyelinating condition, wherein analyzing the physiological parameter comprises measuring reactivity of T cells from the subject to a peptide of myelin basic protein.

26. The method according to claim 25, wherein the myelin basic protein peptide comprises MBP amino acid sequences of 85-99.

27. The method according to claim 10, further comprising administering an additional therapeutic agent selected from the group consisting of: an antibody such as a humanized monoclonal antibody specific to α4-integrin; an enzyme inhibitor such as a type II topoisomerase inhibitor; an antibacterial agent; an antiviral agent; an immunosuppressive agent; a steroid; a nonsteroidal anti-inflammatory agent; an antimetabolite; a cytokine such as an interferon, such as interferon-β; a cytokine blocking agent; an adhesion molecule blocking agent; a soluble cytokine receptor; a sphingosine-1-phosphate receptor modulator such as fingolimod; and a random linear amino acid copolymer composition selected from the group of YEAK (Copaxone®), YFAK, VWAK, and VFAK.

28. The method according to claim 10, wherein the subject is a mouse and, wherein an amount of the fusion protein required to induce tolerance in mice is less than about 1 mg, less than about 500 μg, less than about 300 μg, or less than about 100 μg.

29. The method according to claim 10, wherein an amount of the fusion protein is at least about 10 ng, 100 ng, 1 μg, 50 μg, 100 μg, 150 mg, 200 μg, 250 μg or 300 μg.

30. A method for detecting a presence of a DEC205 receptor in a biological sample, comprising:
contacting a biological sample with the fusion protein of either of claim 1 or 2; and
detecting the fusion protein bound to the DEC205 receptor thereby detecting DEC205 receptor.


SEQUENCE LISTING (82 sequences)

SEQ ID NO: 1 - protein, 15 aa, Artificial Sequence
  The peptide was designed and synthesized
EKPKVEAYKAAAAPA

SEQ ID NO: 2 - protein, 4 aa, Artificial Sequence
  The peptide was designed and synthesized
EKPK

SEQ ID NO: 3 - protein, 4 aa, Artificial Sequence
  The peptide was designed and synthesized
KPKV

SEQ ID NO: 4 - protein, 4 aa, Artificial Sequence
  The peptide was designed and synthesized
PKVE

SEQ ID NO: 5 - protein, 4 aa, Artificial Sequence
  The peptide was designed and synthesized
KVEA

SEQ ID NO: 6 - protein, 4 aa, Artificial Sequence
  The peptide was designed and synthesized
VEAY

SEQ ID NO: 7 - protein, 4 aa, Artificial Sequence
  The peptide was designed and synthesized
EAYK

SEQ ID NO: 8 - protein, 4 aa, Artificial Sequence
  The peptide was designed and synthesized
AYKA

SEQ ID NO: 9 - protein, 4 aa, Artificial Sequence
  The peptide was designed and synthesized
YKAA

SEQ ID NO: 10 - protein, 4 aa, Artificial Sequence
  The peptide was designed and synthesized
KAAA

SEQ ID NO: 11 - protein, 4 aa, Artificial Sequence
  The peptide was designed and synthesized
AAAA

SEQ ID NO: 12 - protein, 4 aa, Artificial Sequence
  The peptide was designed and synthesized
AAAP

SEQ ID NO: 13 - protein, 4 aa, Artificial Sequence
  The peptide was designed and synthesized
AAPA

SEQ ID NO: 14 - protein, 5 aa, Artificial Sequence
  The peptide was designed and synthesized
EKPKV

SEQ ID NO: 15 - protein, 5 aa, Artificial Sequence
  The peptide was designed and synthesized
KPKVE

SEQ ID NO: 16 - protein, 5 aa, Artificial Sequence
  The peptide was designed and synthesized
PKVEA

SEQ ID NO: 17 - protein, 5 aa, Artificial Sequence
  The peptide was designed and synthesized
KVEAY

SEQ ID NO: 18 - protein, 5 aa, Artificial Sequence
  The peptide was designed and synthesized
VEAYK

SEQ ID NO: 19 - protein, 5 aa, Artificial Sequence
  The peptide was designed and synthesized
EAYKA

SEQ ID NO: 20 - protein, 5 aa, Artificial Sequence
  The peptide was designed and synthesized
AYKAA

SEQ ID NO: 21 - protein, 5 aa, Artificial Sequence
  The peptide was designed and synthesized
YKAAA

SEQ ID NO: 22 - protein, 5 aa, Artificial Sequence
  The peptide was designed and synthesized
KAAAA

SEQ ID NO: 23 - protein, 5 aa, Artificial Sequence
  The peptide was designed and synthesized
AAAAP

SEQ ID NO: 24 - protein, 5 aa, Artificial Sequence
  The peptide was designed and synthesized
AAAPA

SEQ ID NO: 25 - protein, 6 aa, Artificial Sequence
  The peptide was designed and synthesized
EKPKVE

SEQ ID NO: 26 - protein, 6 aa, Artificial Sequence
  The peptide was designed and synthesized
KPKVEA

SEQ ID NO: 27 - protein, 6 aa, Artificial Sequence
  The peptide was designed and synthesized
PKVEAY

SEQ ID NO: 28 - protein, 6 aa, Artificial Sequence
  The peptide was designed and synthesized
KVEAYK

SEQ ID NO: 29 - protein, 6 aa, Artificial Sequence
  The peptide was designed and synthesized
VEAYKA

SEQ ID NO: 30 - protein, 6 aa, Artificial Sequence
  The peptide was designed and synthesized
EAYKAA

SEQ ID NO: 31 - protein, 6 aa, Artificial Sequence
  The peptide was designed and synthesized
AYKAAA

SEQ ID NO: 32 - protein, 6 aa, Artificial Sequence
  The peptide was designed and synthesized
YKAAAA

SEQ ID NO: 33 - protein, 6 aa, Artificial Sequence
  The peptide was designed and synthesized
KAAAAP

SEQ ID NO: 34 - protein, 6 aa, Artificial Sequence
  The peptide was designed and synthesized
AAAAPA

SEQ ID NO: 35 - protein, 7 aa, Artificial Sequence
  The peptide was designed and synthesized
EKPKVEA

SEQ ID NO: 36 - protein, 7 aa, Artificial Sequence
  The peptide was designed and synthesized
KPKVEAY

SEQ ID NO: 37 - protein, 7 aa, Artificial Sequence
  The peptide was designed and synthesized
PKVEAYK

SEQ ID NO: 38 - protein, 7 aa, Artificial Sequence
  The peptide was designed and synthesized
KVEAYKA

SEQ ID NO: 39 - protein, 7 aa, Artificial Sequence
  The peptide was designed and synthesized
VEAYKAA

SEQ ID NO: 40 - protein, 7 aa, Artificial Sequence
  The peptide was designed and synthesized
EAYKAAA

SEQ ID NO: 41 - protein, 7 aa, Artificial Sequence
  The peptide was designed and synthesized
AYKAAAA

SEQ ID NO: 42 - protein, 7 aa, Artificial Sequence
  The peptide was designed and synthesized
YKAAAAP

SEQ ID NO: 43 - protein, 7 aa, Artificial Sequence
  The peptide was designed and synthesized
KAAAAPA

SEQ ID NO: 44 - protein, 8 aa, Artificial Sequence
  The peptide was designed and synthesized
EKPKVEAY

SEQ ID NO: 45 - protein, 8 aa, Artificial Sequence
  The peptide was designed and synthesized
KPKVEAYK

SEQ ID NO: 46 - protein, 8 aa, Artificial Sequence
  The peptide was designed and synthesized
PKVEAYKA

SEQ ID NO: 47 - protein, 8 aa, Artificial Sequence
  The peptide was designed and synthesized
KVEAYKAA

SEQ ID NO: 48 - protein, 8 aa, Artificial Sequence
  The peptide was designed and synthesized
VEAYKAAA

SEQ ID NO: 49 - protein, 8 aa, Artificial Sequence
  The peptide was designed and synthesized
EAYKAAAA

SEQ ID NO: 50 - protein, 8 aa, Artificial Sequence
  The peptide was designed and synthesized
AYKAAAAP

SEQ ID NO: 51 - protein, 8 aa, Artificial Sequence
  The peptide was designed and synthesized
YKAAAAPA

SEQ ID NO: 52 - protein, 9 aa, Artificial Sequence
  The peptide was designed and synthesized
EKPKVEAYK

SEQ ID NO: 53 - protein, 9 aa, Artificial Sequence
  The peptide was designed and synthesized
KPKVEAYKA

SEQ ID NO: 54 - protein, 9 aa, Artificial Sequence
  The peptide was designed and synthesized
PKVEAYKAA

SEQ ID NO: 55 - protein, 9 aa, Artificial Sequence
  The peptide was designed and synthesized
KVEAYKAAA

SEQ ID NO: 56 - protein, 9 aa, Artificial Sequence
  The peptide was designed and synthesized
VEAYKAAAA

SEQ ID NO: 57 - protein, 9 aa, Artificial Sequence
  The peptide was designed and synthesized
EAYKAAAAP

SEQ ID NO: 58 - protein, 9 aa, Artificial Sequence
  The peptide was designed and synthesized
AYKAAAAPA

SEQ ID NO: 59 - protein, 10 aa, Artificial Sequence
  The peptide was designed and synthesized
EKPKVEAYKA

SEQ ID NO: 60 - protein, 10 aa, Artificial Sequence
  The peptide was designed and synthesized
KPKVEAYKAA

SEQ ID NO: 61 - protein, 10 aa, Artificial Sequence
  The peptide was designed and synthesized
PKVEAYKAAA

SEQ ID NO: 62 - protein, 10 aa, Artificial Sequence
  The peptide was designed and synthesized
KVEAYKAAAA

SEQ ID NO: 63 - protein, 10 aa, Artificial Sequence
  The peptide was designed and synthesized
VEAYKAAAAP

SEQ ID NO: 64 - protein, 10 aa, Artificial Sequence
  The peptide was designed and synthesized
EAYKAAAAPA

SEQ ID NO: 65 - protein, 11 aa, Artificial Sequence
  The peptide was designed and synthesized
EKPKVEAYKAA

SEQ ID NO: 66 - protein, 11 aa, Artificial Sequence
  The peptide was designed and synthesized
KPKVEAYKAAA

SEQ ID NO: 67 - protein, 11 aa, Artificial Sequence
  The peptide was designed and synthesized
PKVEAYKAAAA

SEQ ID NO: 68 - protein, 11 aa, Artificial Sequence
  The peptide was designed and synthesized
KVEAYKAAAAP

SEQ ID NO: 69 - protein, 11 aa, Artificial Sequence
  The peptide was designed and synthesized
VEAYKAAAAPA

SEQ ID NO: 70 - protein, 12 aa, Artificial Sequence
  The peptide was designed and synthesized
EKPKVEAYKAAA

SEQ ID NO: 71 - protein, 12 aa, Artificial Sequence
  The peptide was designed and synthesized
KPKVEAYKAAAA

SEQ ID NO: 72 - protein, 12 aa, Artificial Sequence
  The peptide was designed and synthesized
PKVEAYKAAAAP

SEQ ID NO: 73 - protein, 12 aa, Artificial Sequence
  The peptide was designed and synthesized
KVEAYKAAAAPA

SEQ ID NO: 74 - protein, 13 aa, Artificial Sequence
  The peptide was designed and synthesized
EKPKVEAYKAAAA

SEQ ID NO: 75 - protein, 13 aa, Artificial Sequence
  The peptide was designed and synthesized
KPKVEAYKAAAAP

SEQ ID NO: 76 - protein, 13 aa, Artificial Sequence
  The peptide was designed and synthesized
PKVEAYKAAAAPA

SEQ ID NO: 77 - protein, 14 aa, Artificial Sequence
  The peptide was designed and synthesized
EKPKVEAYKAAAAP

SEQ ID NO: 78 - protein, 14 aa, Artificial Sequence
  The peptide was designed and synthesized
KPKVEAYKAAAAPA

SEQ ID NO: 79 - protein, 1924 aa, Artificial Sequence
  The peptide was designed and synthesized
MGWSCIILFLVATATGVHSQVQIEFALGKPIPNPLLGLDSTSRSGRAANDPFTIVHGNTGKCIKPVYGWIVADDCDETEDKLWKWVSQHRLFHLHSQKCLGLDITKSVNELRMFSCDSSAMLWWKCEHHSLYGAARYRLALKDGHGTAISNASDVWKKGGSEESLCDQPYHEIYTRDGNSYGRPCEFPFLIDGTWHHDCILDEDHSGPWCATTLNYEYDRKWGICLKPENGCEDNWEKNEQFGSCYQFNTQTALSWKEAYVSCQNQGADLLSINSAAELTYLKEKEGIAKIFWIGLNQLYSARGWEWSDHKPLNFLNWDPDRPSAPTIGGSSCARMDAESGLWQSFSCEAQLPYVCRKPLNNTVELTDVWTYSDTRCDAGWLPNNGFCYLLVNESNSWDKAHAKCKAFSSDLISIHSLADVEVVVTKLHNEDIKEEVWIGLKNINIPTLFQWSDGTEVTLTYWDENEPNVPYNKTPNCVSYLGELGQWKVQSCEEKLKYVCKRKGEKLNDASSDKMCPPDEGWKRHGETCYKIYEDEVPFGTNCNLTITSRFEQEYLNDLMKKYDKSLRKYFWTGLRDVDSCGEYNWATVGGRRRAVTFSNWNFLEPASPGGCVAMSTGKSVGKWEVKDCRSFKALSICKKMSGPLGPEEASPKPDDPCPEGWQSFPASLSCYKVFHAERIVRKRNWEEAERFCQALGAHLSSFSHVDEIKEFLHFLTDQFSGQHWLWIGLNKRSPDLQGSWQWSDRTPVSTIIMPNEFQQDYDIRDCAAVKVFHRPWRRGWHFYDDREFIYLRPFACDTKLEWVCQIPKGRTPKTPDWYNPDRAGIHGPPLIIEGSEYWFVADLHLNYEEAVLYCASNHSFLATITSFVGLKAIKNKIANISGDGQKWWIRISEWPIDDHFTYSRYPWHRFPVTFGEECLYMSAKTWLIDLGKPTDCSTKLPFICEKYNVSSLEKYSPDSAAKVQCSEQWIPFQNKCFLKIKPVSLTFSQASDTCHSYGGTLPSVLSQIEQDFITSLLPDMEATLWIGLRWTAYEKINKWTDNRELTYSNFHPLLVSGRLRIPENFFEEESRYHCALILNLQKSPFTGTWNFTSCSERHFVSLCQKYSEVKSRQTLQNASETVKYLNNLYKIIPKTLTWHSAKRECLKSNMQLVSITDPYQQAFLSVQALLHNSSLWIGLFSQDDELNFGWSDGKRLHFSRWAETNGQLEDCVVLDTDGFWKTVDCNDNQPGAICYYSGNETEKEVKPVDSVKCPSPVLNTPWIPFQNCCYNFIITKNRHMATTQDEVHTKCQKLNPKSHILSIRDEKENNFVLEQLLYFNYMASWVMLGITYRNNSLMWFDKTPLSYTHWRAGRPTIKNEKFLAGLSTDGFWDIQTFKVIEEAVYFHQHSILACKIEMVDYKEEHNTTLPQFMPYEDGIYSVIQKKVTWYEALNMCSQSGGHLASVHNQNGQLFLEDIVKRDGFPLWVGLSSHDGSESSFEWSDGSTFDYIPWKGQTSPGNCVLLDPKGTWKHEKCNSVKDGAICYKPTKSKKLSRLTYSSRCPAAKENGSRWIQYKGHCYKSDQALHSFSEAKKLCSKHDHSATIVSIKDEDENKFVSRLMRENNNITMRVWLGLSQHSVDQSWSWLDGSEVTFVKWENKSKSGVGRCSMLIASNETWKKVECEHGFGRVVCKVPLGPDSSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSSSQLSR

SEQ ID NO: 80 - protein, 13 aa, Artificial Sequence
  The peptide was designed and synthesized
HSLGKWLGHPNKF

SEQ ID NO: 81 - protein, 1939 aa, Artificial Sequence
  The peptide was designed and synthesized
MGWSCIILFLVATATGVHSQVQIEFALGKPIPNPLLGLDSTSRSGRAANDPFTIVHGNTGKCIKPVYGWIVADDCDETEDKLWKWVSQHRLFHLHSQKCLGLDITKSVNELRMFSCDSSAMLWWKCEHHSLYGAARYRLALKDGHGTAISNASDVWKKGGSEESLCDQPYHEIYTRDGNSYGRPCEFPFLIDGTWHHDCILDEDHSGPWCATTLNYEYDRKWGICLKPENGCEDNWEKNEQFGSCYQFNTQTALSWKEAYVSCQNQGADLLSINSAAELTYLKEKEGIAKIFWIGLNQLYSARGWEWSDHKPLNFLNWDPDRPSAPTIGGSSCARMDAESGLWQSFSCEAQLPYVCRKPLNNTVELTDVWTYSDTRCDAGWLPNNGFCYLLVNESNSWDKAHAKCKAFSSDLISIHSLADVEVVVTKLHNEDIKEEVWIGLKNINIPTLFQWSDGTEVTLTYWDENEPNVPYNKTPNCVSYLGELGQWKVQSCEEKLKYVCKRKGEKLNDASSDKMCPPDEGWKRHGETCYKIYEDEVPFGTNCNLTITSRFEQEYLNDLMKKYDKSLRKYFWTGLRDVDSCGEYNWATVGGRRRAVTFSNWNFLEPASPGGCVAMSTGKSVGKWEVKDCRSFKALSICKKMSGPLGPEEASPKPDDPCPEGWQSFPASLSCYKVFHAERIVRKRNWEEAERFCQALGAHLSSFSHVDEIKEFLHFLTDQFSGQHWLWIGLNKRSPDLQGSWQWSDRTPVSTIIMPNEFQQDYDIRDCAAVKVFHRPWRRGWHFYDDREFIYLRPFACDTKLEWVCQIPKGRTPKTPDWYNPDRAGIHGPPLIIEGSEYWFVADLHLNYEEAVLYCASNHSFLATITSFVGLKAIKNKIANISGDGQKWWIRISEWPIDDHFTYSRYPWHRFPVTFGEECLYMSAKTWLIDLGKPTDCSTKLPFICEKYNVSSLEKYSPDSAAKVQCSEQWIPFQNKCFLKIKPVSLTFSQASDTCHSYGGTLPSVLSQIEQDFITSLLPDMEATLWIGLRWTAYEKINKWTDNRELTYSNFHPLLVSGRLRIPENFFEEESRYHCALILNLQKSPFTGTWNFTSCSERHFVSLCQKYSEVKSRQTLQNASETVKYLNNLYKIIPKTLTWHSAKRECLKSNMQLVSITDPYQQAFLSVQALLHNSSLWIGLFSQDDELNFGWSDGKRLHFSRWAETNGQLEDCVVLDTDGFWKTVDCNDNQPGAICYYSGNETEKEVKPVDSVKCPSPVLNTPWIPFQNCCYNFIITKNRHMATTQDEVHTKCQKLNPKSHILSIRDEKENNFVLEQLLYFNYMASWVMLGITYRNNSLMWFDKTPLSYTHWRAGRPTIKNEKFLAGLSTDGFWDIQTFKVIEEAVYFHQHSILACKIEMVDYKEEHNTTLPQFMPYEDGIYSVIQKKVTWYEALNMCSQSGGHLASVHNQNGQLFLEDIVKRDGFPLWVGLSSHDGSESSFEWSDGSTFDYIPWKGQTSPGNCVLLDPKGTWKHEKCNSVKDGAICYKPTKSKKLSRLTYSSRCPAAKENGSRWIQYKGHCYKSDQALHSFSEAKKLCSKHDHSATIVSIKDEDENKFVSRLMRENNNITMRVWLGLSQHSVDQSWSWLDGSEVTFVKWENKSKSGVGRCSMLIASNETWKKVECEHGFGRVVCKVPLGPDSSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSSSQLSREKPKVEAYKAAAAPA

SEQ ID NO: 82 - protein, 1937 aa, Artificial Sequence
  The peptide was designed and synthesized
MGWSCIILFLVATATGVHSQVQIEFALGKPIPNPLLGLDSTSRSGRAANDPFTIVHGNTGKCIKPVYGWIVADDCDETEDKLWKWVSQHRLFHLHSQKCLGLDITKSVNELRMFSCDSSAMLWWKCEHHSLYGAARYRLALKDGHGTAISNASDVWKKGGSEESLCDQPYHEIYTRDGNSYGRPCEFPFLIDGTWHHDCILDEDHSGPWCATTLNYEYDRKWGICLKPENGCEDNWEKNEQFGSCYQFNTQTALSWKEAYVSCQNQGADLLSINSAAELTYLKEKEGIAKIFWIGLNQLYSARGWEWSDHKPLNFLNWDPDRPSAPTIGGSSCARMDAESGLWQSFSCEAQLPYVCRKPLNNTVELTDVWTYSDTRCDAGWLPNNGFCYLLVNESNSWDKAHAKCKAFSSDLISIHSLADVEVVVTKLHNEDIKEEVWIGLKNINIPTLFQWSDGTEVTLTYWDENEPNVPYNKTPNCVSYLGELGQWKVQSCEEKLKYVCKRKGEKLNDASSDKMCPPDEGWKRHGETCYKIYEDEVPFGTNCNLTITSRFEQEYLNDLMKKYDKSLRKYFWTGLRDVDSCGEYNWATVGGRRRAVTFSNWNFLEPASPGGCVAMSTGKSVGKWEVKDCRSFKALSICKKMSGPLGPEEASPKPDDPCPEGWQSFPASLSCYKVFHAERIVRKRNWEEAERFCQALGAHLSSFSHVDEIKEFLHFLTDQFSGQHWLWIGLNKRSPDLQGSWQWSDRTPVSTIIMPNEFQQDYDIRDCAAVKVFHRPWRRGWHFYDDREFIYLRPFACDTKLEWVCQIPKGRTPKTPDWYNPDRAGIHGPPLIIEGSEYWFVADLHLNYEEAVLYCASNHSFLATITSFVGLKAIKNKIANISGDGQKWWIRISEWPIDDHFTYSRYPWHRFPVTFGEECLYMSAKTWLIDLGKPTDCSTKLPFICEKYNVSSLEKYSPDSAAKVQCSEQWIPFQNKCFLKIKPVSLTFSQASDTCHSYGGTLPSVLSQIEQDFITSLLPDMEATLWIGLRWTAYEKINKWTDNRELTYSNFHPLLVSGRLRIPENFFEEESRYHCALILNLQKSPFTGTWNFTSCSERHFVSLCQKYSEVKSRQTLQNASETVKYLNNLYKIIPKTLTWHSAKRECLKSNMQLVSITDPYQQAFLSVQALLHNSSLWIGLFSQDDELNFGWSDGKRLHFSRWAETNGQLEDCVVLDTDGFWKTVDCNDNQPGAICYYSGNETEKEVKPVDSVKCPSPVLNTPWIPFQNCCYNFIITKNRHMATTQDEVHTKCQKLNPKSHILSIRDEKENNFVLEQLLYFNYMASWVMLGITYRNNSLMWFDKTPLSYTHWRAGRPTIKNEKFLAGLSTDGFWDIQTFKVIEEAVYFHQHSILACKIEMVDYKEEHNTTLPQFMPYEDGIYSVIQKKVTWYEALNMCSQSGGHLASVHNQNGQLFLEDIVKRDGFPLWVGLSSHDGSESSFEWSDGSTFDYIPWKGQTSPGNCVLLDPKGTWKHEKCNSVKDGAICYKPTKSKKLSRLTYSSRCPAAKENGSRWIQYKGHCYKSDQALHSFSEAKKLCSKHDHSATIVSIKDEDENKFVSRLMRENNNITMRVWLGLSQHSVDQSWSWLDGSEVTFVKWENKSKSGVGRCSMLIASNETWKKVECEHGFGRVVCKVPLGPDSSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSSSQLSRHSLGKWLGHPNKF